(19) United States
(12) Patent Application Publication
(10) Pub. No.: US 2021/0349065 A1
(43) Pub. Date: Nov. 11, 2021

(54) DIRECT CAPTURE SUBSTRATE, DEVICE AND METHOD

(71) Applicant: EMISSOL LLC, Mill Creek, WA (US)

(72) Inventors: Mansour MASOUDI, Mill Creek, WA (US); Edward Benjamin TEGELER, IV, Shoreline, WA (US); Jacob Roy HENSEL, Bothel, WA (US)

(21) Appl. No.: 17/317,819

(22) Filed: May 11, 2021

Related U.S. Application Data
(60) Provisional application No. 63/022,965, filed on May 11, 2020.
(60) Provisional application No. 63/023,011, filed on May 11, 2020.
(60) Provisional application No. 63/022,798, filed on May 11, 2020.

(51) Int. Cl. G01N 33/00; G01N 1/02; G01F 1/00

(52) U.S. Cl. CPC G01N 33/004; G01F 1/002; G01N 1/02

(57) ABSTRACT

A capture device comprising a capture device substrate with non-linear flow channels, a method of manufacturing the capture device and a method of using the capture device including to capture CO₂ from the air is disclosed.

### RELATED APPLICATIONS

This application claims the benefit of U.S. Provisional Application Ser. No. 63/022,965 filed May 11, 2020; and U.S. Provisional Application Ser. No. 63/022,798 filed May 11, 2020; and U.S. Provisional Application Ser. No. 63/023,011 filed May 11, 2020, the disclosures of which are incorporated by reference herein in their entirety.

### STATEMENT OF GOVERNMENT SPONSORSHIP

The present invention was partly made with funding from the US Department of Energy under grant No. DE-SC0015946. The US Government may have certain rights to this invention.

### FIELD

The instant disclosure is generally directed to substrates and devices for treatment of a fluid. In particular, the instant disclosure is directed to so-called direct air capture substrates suitable for use treatment of a fluid by removal of a material present in the fluid by absorption, adsorptions, sequestering, containment, and/or by chemical reaction resulting in treatment of a fluid flowing through the substrate, and the like.

### BACKGROUND

Direct Air Capture (DAC) is typically conducted using some sort of substrate coated with an adsorbent or absorbent to adsorb CO₂, followed by desorbing and releasing CO₂ periodically. The substrate preferably has a large amount of ‘surface area’ per unit area ideal for CO₂ adsorption/desorption while yielding very low pressure drop and hence reducing the power consumption required to pump the air or other fluid to be treated through the device.

As shown in prior art FIG. 1, conventional capture substrates comprise a plurality of straight channels through which air (or in general any fluid) flows through. This fluid flow is typically at a Reynolds number or other such index in the regime of laminar flow (due to operational needs to sustain its low pressure drop or flow resistance resulting in straight streamlines as opposed to turbulent flow. In such a flow, for CO₂ to be adsorbed to the sorbent-coated walls or by a sorbent present along the walls of the channel, the CO₂ species must travel across the flow streamlines driven by diffusion resulting from higher CO₂ concentration at the channel flow centerline vs. its lower concentration near the channel wall. The base flow or convection flow itself has essentially no role in CO₂ transport from the fluid being treated into the sorbent. Diffusion, the dominant process for straight channels is known to be a slow process as compared to convection and other motive forces present.

The capture substrate, e.g., a honeycomb or other arrangement, also comes with significant barriers to use including the cost due to the energy required to pump or otherwise draw the air through the capture substrate due to the need to overcome the backpressure or resistance due to air passing through the channels, as well as the power requirements in the form of electrical heating or steam, and/or pressure required to switch the CO₂ adsorption process into a desorption or a separation process to effectively capture CO₂.

There is a need in the art to improve the contactor substrate and process useful in DAC and/or other fluid treatment devices.

### SUMMARY

In embodiments, a capture device substrate comprises a fluid inlet in fluid communication with a fluid outlet through at least one flow channel disposed along at least one flow path disposed within a body of the substrate; each flow channel comprising a cross-sectional shape comprising a plurality of sides defining a cross-sectional area, determined orthogonal to the flow path; at least a portion of the flow path comprising an essentially sinusoidal shape, an essentially helical shape, or a combination thereof, configured to produce one or more stable Dean vortical structures in a fluid flowing through the flow channel when determined at a Reynolds number from about 100 to 500. In embodiments, the capture device substrate comprises a sorbent effective to absorb, adsorb, sequester, and/or undergo a chemical reaction with one or more components present in the fluid flowing through at least a portion of the flow channel.

In one or more embodiments, a fluid treatment device comprises a capture device substrate according to one or more embodiments disclosed herein.

In one or more embodiments, a method of treating a fluid comprises the steps of directing a fluid comprising a first concentration of a target compound through a fluid treatment device comprising a capture device substrate according to one or more embodiments disclosed herein to produce a treated fluid having a second concentration of the target compound which is less than the first concentration. In an embodiment, the method further comprises a desorption step wherein the target compound is released and recovered. Preferably, the fluid is air and the target compound is or includes carbon dioxide.

### BRIEF DESCRIPTION OF THE DRAWINGS

FIG. 1 shows a prior art linear absorption channel;

FIG. 2 is side view of a prior art substrate having a linear flow channel coated with a sorbent;

FIG. 3 is a perspective view of a prior art substrate having inlet and outlet linear flow channels separated by a porous sidewall;

FIG. 4 is a perspective view of an essentially helical flow channel according to embodiments disclosed herein;

FIG. 5 is a perspective view of essentially helical flow channel according to embodiments disclosed herein;

FIG. 6 is a side view of an essentially sinusoidal flow channel according to embodiments disclosed herein;

FIG. 7 is a diagram showing a curved flow channel and the Deanvortical structures produced within the base flow of a fluid flowing through the flow channel according to embodiments disclosed herein;

FIG. 8 is a fluid flow depiction of Dean vortices produced by the curved flow channel shown in FIG. 7.

FIG. 9 is a direct capture treatment device having a capture device substrate comprising essentially helical flow channels according to embodiments disclosed herein;

FIG. 10 is a direct capture treatment device having a capture device substrate comprising essentially sinusoidal flow channels according to embodiments disclosed herein;

FIG. 11 is a direct capture treatment device treatment having a capture device substrate comprising essentially helical-essentially sinusoidal flow channels according to embodiments disclosed herein;

FIG. 12 is a fluid treatment device treatment having a capture device substrate comprising essentially sinusoidal-essentially helical flow channels according to embodiments disclosed herein;

FIG. 13 is a side view of the essentially sinusoidal-essentially helical flow channels shown in FIG. 12 according to embodiments disclosed herein;

FIG. 14A is a solid view of a plurality of essentially sinusoidal flow channels having a square cross-sectional shape according to embodiments disclosed herein;

FIG. 14B is a transparent view of the essentially sinusoidal flow channels shown in FIG. 14A according to embodiments disclosed herein;

FIG. 15 is a transparent view of a plurality of sinusoidal inlet flow channels having a square cross-sectional shape disposed within a common outlet flow channel or collector having a circular cross-sectional shape according to embodiments disclosed herein;

FIG. 16A is a solid view of a plurality of essentially helical flow channels having a square cross-sectional shape according to embodiments disclosed herein;

FIG. 16B is a transparent view of the embodiment shown in FIG. 16A;

FIG. 17A is a transparent view of a plurality of essentially helical flow channels having a square cross-sectional shape according to embodiments disclosed herein;

FIG. 17B is a transparent view of a plurality of essentially helical flow channels having a square cross-sectional shape according to embodiments disclosed herein;

FIG. 18A is a solid view of a sinusoidal inlet flow channel having a square cross-sectional shape disposed within an outlet flow channel having a circular cross-sectional shape according to embodiments disclosed herein;

FIG. 18B is a transparent view of the embodiment shown in FIG. 18A according to embodiments disclosed herein;

FIG. 19A is a block diagram of a direct capture device according to embodiments disclosed herein;

FIG. 19B is a block diagram of a direct capture substrate shown in FIG. 19A according to an embodiment disclosed herein;

FIG. 19C is a block diagram of a direct capture substrate shown in FIG. 19A according to an embodiment disclosed herein;

FIG. 20 is a plot of Sherwood number vs Reynolds number of flow channels according to embodiments disclosed herein and linear flow channels;

FIG. 21 is a plot of pumping power vs Reynolds number of flow channels along with capture efficiency according to embodiments disclosed herein; and

FIG. 22 is a partial perspective drawing of a capture device substrate comprising an essentially sinusoidal flow channel in which a liquid sorbent is directed through the flow channel in a counter flow direction according to embodiments disclosed herein;

FIG. 23 is a portion of the fluid flow channel shown in FIG. 22;

FIG. 24 is a partial perspective drawing of a concentric essentially helical channel capture device substrate according to embodiments disclosed herein, formed from metal sheets, plastic sheets, or both according to embodiments disclosed herein;

FIG. 25 is a top perspective view of a concentric essentially helical channel capture device substrate according to embodiments disclosed herein;

FIG. 26 is a side perspective view of two essentially helical flow channels in a nested arrangement according to embodiments disclosed herein;

FIG. 27 is a top down perspective view of two essentially helical flow channels in a nested arrangement according to alternative embodiments disclosed herein;

FIG. 28 is a top view of two essentially helical flow channels in a nested arrangement having a common sidewall according to embodiments disclosed herein;

FIG. 29 is a top down perspective view of an essentially helical flow channel having a circular cross-sectional shape according to embodiments disclosed herein;

FIG. 30 is a perspective view of a plurality of the flow channels disposed in a capture device substrate according to embodiments disclosed herein;

FIG. 31A is a top view of a flow channel having a circular cross-sectional shape according to embodiments disclosed herein;

FIG. 31B is a top view of a plurality of the flow channels shown in FIG. 31A arranged within a capture device substrate with minimum wasted space and having common walls between the channels according to embodiments disclosed herein;

FIG. 32A is a top view of a flow channel having a hexagonal cross-sectional shape according to embodiments disclosed herein;

FIG. 32B is a top view of a plurality of the flow channels shown in FIG. 32A arranged within a capture device substrate with minimum wasted space having common walls between the channels according to embodiments disclosed herein;

FIG. 33A is a top down perspective view of a flow channel having a hexagonal cross-sectional shape according to embodiments disclosed herein;

FIG. 33B is a top down perspective view of a plurality of the flow channels shown in FIG. 33A arranged within a capture device substrate with minimum wasted space having common walls between the channels according to embodiments disclosed herein;

FIG. 34A is a top view of a flow channel having a square cross-sectional shape according to embodiments disclosed herein;

FIG. 34B is a top view of a plurality of the flow channels shown in FIG. 34A arranged within a capture device substrate with minimum wasted space having common walls between the channels according to embodiments disclosed herein;

FIG. 35A is a top view of a flow channel having a triangular cross-sectional shape according to embodiments disclosed herein;

FIG. 35B is a top view of a plurality of the flow channels shown in FIG. 35A arranged within a capture device substrate with minimum wasted space having common walls between the channels according to embodiments disclosed herein;

FIG. 36A is a top view of a flow channel having a hexagonal cross-sectional shape showing the maximum and minimum radius of the flow channels according to embodiments disclosed herein; and

FIG. 36B is a plot showing how the flow channel radius periodically varies along the center axis of the flow channel as determined by the distance along the flow channel center axis from the top of the substrate body;

FIG. 37 is a solid view of a plurality of helical flow channels having a square cross-sectional shape according to embodiments disclosed herein;

FIG. 38 is a transparent view of a helical inlet flow channel having a square cross-sectional shape coaxially disposed within an outlet flow channel having a circular cross-sectional shape according to embodiments disclosed herein;

FIG. 39 is a transparent view of a plurality of conical inlet flow channels having a square cross-sectional shape longitudinally disposed within a single or common outlet flow channel having a circular cross-sectional shape according to embodiments disclosed herein;

FIG. 40 is a transparent view of a sinusoidal inlet flow channel having a square cross-sectional shape disposed concentrically within an out-flow channel having a hexagonal cross-sectional shape according to embodiments disclosed herein;

FIG. 41 is a transparent view of a plurality of sinusoidal inlet flow channels having a square cross-sectional shape disposed within a common outlet flow channel or collector having a circular cross-sectional shape according to embodiments disclosed herein;

FIG. 42A is a graph showing comparison of a model and experimental results of outlet CO₂ concentration for an embodiment disclosed herein;

FIG. 42B is a graph showing comparison of a model and experimental results of outlet CO₂ concentration for an embodiment disclosed herein;

FIG. 42C is a graph showing comparison of a model and experimental results of outlet CO₂ concentration for an embodiment disclosed herein;

FIG. 42D is a graph showing comparison of a model and experimental results of outlet CO₂ concentration for an embodiment disclosed herein;

FIG. 43 is a graph showing CO₂ capture rate normalized by volume and sorbent mass as a function of increasing Sherwood number according to embodiments disclosed herein; and

FIG. 44 is a graph showing desorption curves for resistance and convective heating methods of substrates according to embodiments disclosed herein.

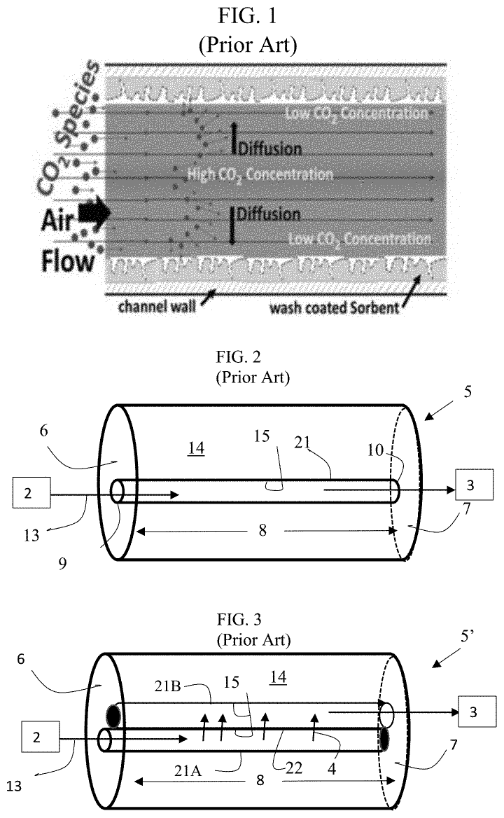

### DETAILED DESCRIPTION

At the outset, it should be noted that in the development of any such actual embodiment, numerous implementation-specific decisions must be made to achieve the developer's specific goals, such as compliance with system related and business related constraints, which will vary from one implementation to another. Moreover, it will be appreciated that such a development effort might be complex and time consuming but would nevertheless be a routine undertaking for those of ordinary skill in the art having the benefit of this disclosure. In addition, the composition used/disclosed herein can also comprise some components other than those cited.

In the summary and this detailed description, each numerical value should be read once as modified by the term “about” (unless already expressly so modified), and then read again as not so modified unless otherwise indicated in context. Also, in the summary and this detailed description, it should be understood that a physical range listed or described as being useful, suitable, or the like, is intended that any and every value within the range, including the end points, is to be considered as having been stated. For example, “a range of from 1 to 10” is to be read as indicating each and every possible number along the continuum between about 1 and about 10. Thus, even if specific data points within the range, or even no data points within the range, are explicitly identified or refer to only a few specific, it is to be understood that inventors appreciate and understand that any and all data points within the range are to be considered to have been specified, and that inventors possessed knowledge of the entire range and all points within the range.

The following definitions are provided in order to aid those skilled in the art in understanding the detailed description, listed embodiments, and the appended claims.

As used in the specification and claims, “near” is inclusive of “at.” For purposes herein, a capture device substrate may also be referred to interchangeably as capture device substrate, a capture substrate, a honeycomb, a contactor, or simply as a substrate.

As shown by way of example in prior art FIG. 2, a capture device substrate, generally referred to as 5, includes an inlet end 6 separated from an outlet end 7 by a body length 8 wherein the inlet end 6 is in fluid communication with the outlet end 7 through at least one flow channel 21 disposed through the body 14 of the substrate 5. Substrates according to one or more embodiments of the instant disclosure may further comprise a sorbent 15 disposed in or present on a wall of the channel 21 suitable for removing CO₂ or other materials from a fluid 13 flowing through the channel 21 from an inlet opening 9 to and exiting at outlet opening 10.

Accordingly, the capture device substrate according to embodiments of the instant disclosure includes a fluid inlet 2 in fluid communication with a fluid outlet 3 through at least one flow channel 21 disposed along at least one flow path disposed within a body.

In other embodiments, again as shown by way of example in prior art FIG. 3, the capture device substrate 5′ includes a fluid inlet which is in indirect fluid communication with the fluid outlet via fluid communication between a first flow channel 21A and the second flow channel 21B, wherein at least a portion of the first and second flow channels have a porosity or other means (4) by which fluid communication is achieved.

For purposes herein the capture device substrates are not so limited to absorption or adsorption of an analyte, but they may be, or may also be suitable for conducting chemical reactions with the analyte present in the fluid flowing therethrough, e.g., the sorbent may be or may include a catalytic component disposed on or in a wall of the flow channel. Accordingly, the capture device substrates according to the instant disclosure may be used to accomplish other physical processes such as filtering, reactive filtering, heat transfer, chemical conversion or synthesis, and/or the like.

As shown by way of example in prior art FIG. 3, a capture device substrate may include a flow channel plugged at one end in fluid communication through a sidewall of the flow channel with another flow channel which is plugged on another end of the substrate body. Accordingly, in embodiments the capture device substrate may comprise a first flow channel 21A disposed proximate to a second flow channel 21B, wherein at least a portion of at least one side of the first flow channel forms at least one common sidewall 22 between at least a portion of at least one side of the second flow channel, wherein at least a portion of the at least one common sidewall comprises a porosity, a conduit, a via, or a combination thereof, wherein the fluid inlet is in fluid communication with the fluid outlet through at least a portion of the at least one common sidewall 22.

By way of example, a capture device substrate as shown in FIG. 3 has a first channel 21A blocked as indicated by the filled portion of the channel, on the outlet end 7 of the body 14 which is in direct fluid communication with the fluid inlet 2, but which is not in direct fluid communication with the fluid outlet 3. Instead the fluid inlet 2 is in indirect fluid communication via fluid communication between the first flow channel 21A and the second flow channel 21B as indicated by arrows 4 only one of which is labeled for clarity. Likewise, the second flow channel 21B is in directed fluid communication with the fluid outlet 3 but is in indirect fluid communication with the fluid inlet 2.

It is to be understood that for purposes herein, discussion of a fluid flowing through a capture device substrate refers to the fluid flow having a mass flowrate, a pressure, a temperature and under conditions consistent with the intended purpose of the capture device substrate. For example, fluid flow through a capture device substrate employed for direct air capture of CO₂ for treatment of ambient air may be at a first set of conditions having a mass flow rate, a temperature, and under conditions consisting with DAC, while treatment of an exhaust stream generated by combustion or some other source refers to a fluid flow and composition having a mass flow rate, a temperature, and under conditions consisting with a typical exhaust stream as readily understood by one of minimal skill in the art.

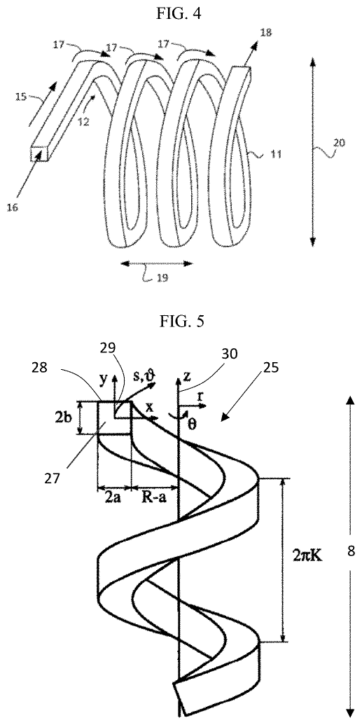

For purposes herein, as shown in FIGS. 4 and 5, a channel having an essentially helical flow path 11 conforms to the general description of a helix, being a curve in 3-dimensional space, which may be described in Cartesian coordinates according to the equations: x(t)=cos(t); y(t)=sin(t); z(t)=t, wherein as the parameter t increases, the point (x(t),y(t),z(t)) traces a right-handed helix of pitch 2π (or slope 1) and radius 1 about the z-axis, in a right-handed coordinate system. Likewise, in cylindrical coordinates (r, θ, h), the same helix is parametrized by r(t)=1; θ(t)=t; and h(t)=t. A circular helix of radius “a” (half of diameter 20) and slope b/a (indicated as 19), or pitch 2πb, is described by x(t)=a cos(t); y(t)=a sin(t); z(t)=bt.

It is to be understood that for purposes herein, a channel having an “essentially” helical shape or channel flow path refers to a channel that is generally represented by a helix. Accordingly, for purposes herein it is to be understood that an essentially helical shape includes a helical shape. However, the channel need not be strictly defined by a helix, but may approximate a helix as would be readily understood by one of minimal skill in the art. In addition, a channel having an “essentially” helical shape according to the instant disclosure includes a shape that results from a mathematical superposition, transform, or other mathematical operation of two or more essentially helical, which for purposes herein includes helical) shapes and/or an essentially helical shape with another shape.

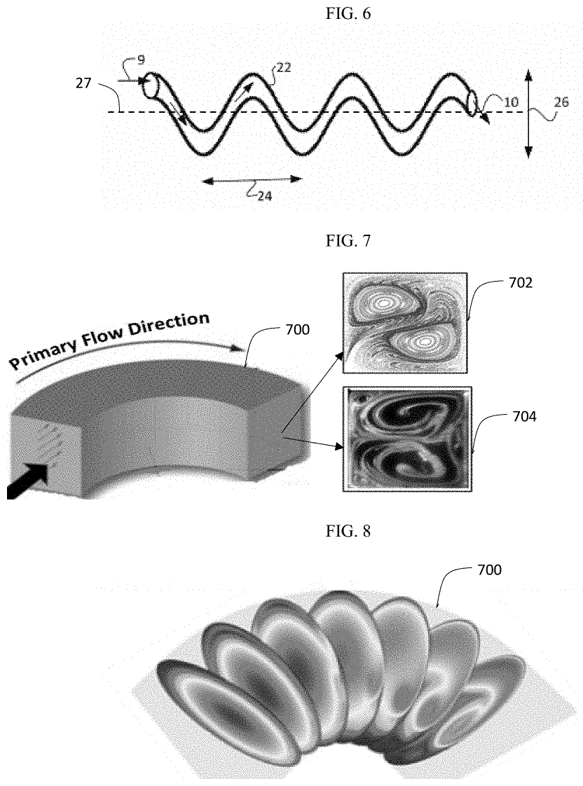

For purposes herein, as shown in FIG. 6, a flow channel having a flow path with an essentially sinusoidal shape (an essentially sinusoidal flow channel) refer to a flow channels having a shape which is essentially described by the mathematical sine function, i.e., a sine wave or sinusoid, according to the mathematical sine function.

However, the channel need not be strictly defined by a sine wave or sinusoid, and for purposes herein includes a shape defined by a periodic oscillation, preferably a smooth periodic oscillation, having a wavelength 24 and an amplitude 26 between a minimum and a maximum about a center axis 27 as shown in FIG. 6, according to understanding common to the skilled artisan. For purposes herein, an essentially sinusoidal shape includes shapes that approximate a sine wave or sinusoid as would be readily understood by one of minimal skill in the art. Other similar descriptions of an essentially sinusoidal shape include “wavy” or wave-like, herringbone, pseudo essentially sinusoidal, pseudo wavy, saw tooth, stepped, serpentine, and/or variations and combination thereof. In addition, a channel having an “essentially” sinusoidal shape according to the instant disclosure includes a shape that results from a mathematical superposition, transform, or other mathematical operation of two or more essentially sinusoidal shapes and/or an essentially sinusoidal shape with another shape. Accordingly, for purposes herein it is to be understood that an essentially sinusoidal shape includes a sinusoidal shape.

For purposes herein, arrangement of channels within the substrate body refers to the centerline of the channel flow path, which is the locust of points defined by the geometric center of each cross-section of the channel determined orthogonal to a center axis of the channel at each point from the inlet of the body to the outlet of the body, i.e., along the length of the substrate. Accordingly, the centerline of the channel need not be the geometric center of the substrate body, and is independent of the overall shape of the substrate body. For example, if a substrate body is linear from the inlet to the outlet, the channel flow path may be defined by a shape along a longitudinal axis of the substrate body from the inlet to the outlet along the length of the body. However, if the substrate body is curved or has a U-shape, the channel flow path need not be along a longitudinal axis of the substrate body, but may follow along any line connecting the inlet of the substrate body to the outlet of the substrate body that is disposed within the substrate body.

For purposes herein, a flow channel may have a single inlet, multiple inlets, a single outlet, multiple outlets, or any combination thereof.

For purposes herein a flow channel disposed along a flow path having a particular shape, for brevity may be referred to by that shaped flow channel. For example, a flow channel disposed and/or oriented along a flow path having an essentially sinusoidal shape may be referred to herein simply as an essentially sinusoidal flow channel.

For purposes herein, a direct capture substrate which is formed from, and/or which comprises a thermoplastic polymer, a thermoset polymer, and/or any combination thereof, for brevity may be simply referred to as comprising a “plastic”, unless specifically stated otherwise.

For purposes herein it is to be understood that reference to a Dean vortical structure refers to a flow having secondary flow patterns comprising one or more vortex or vortex like structures. While applicant realizes that there is a general agreement among those of skill in the art that Dean vortical structures form in essentially helical flow paths, there is debate as to the name given to the vortical structures that form in essentially sinusoidal flow paths. Accordingly for purposes herein, reference to the presence of a Dean vortical structure includes the formation of other types of stable vortical structures including Taylor, Goertler (Gortler), Taylor-Gortler, and the like, that form in flows flowing through an essentially sinusoidal flow channel. Accordingly, for purposes herein, it is to be understood that disclosure and/or recitation of the presence of a stable Dean vortical structure indicates the presence of a stable secondary flow within the base flow flowing through the flow channel. In other terms, reference to a stable Dean vortical structure refers to a stable Dean-like vortical structure and/or a stable essentially Dean vortical structure.

For purposes herein, the ability of a flow channel disposed along a flow path comprising an essentially helical and/or an essentially sinusoidal shape configured to produce one or more stable Dean vortical structures in a fluid flowing through the flow channel is determined at a Reynolds number from about 100 to 500. This range of Reynolds numbers is selected for purposes herein to represent a non-turbulent flow range, and is utilized to define the test conditions for the formation of stable Dean vortical structures in the fluid flow channel according to the summary, figures, description and claims of the instant disclosure. The presence of stable Dean vortical structures may be determined experimentally, by modeling, or any combination or by any method known in the art, so long as they are determined at a flow rate through the flow channel representing a Reynolds number from about 100 to 500 for the fluid. It is to be understood that in an intended use, flows through the capture device substrate may be at a higher or lower Reynolds number than this value, but for purposes herein, and the claims recited herein, the ability of a capture device substrate to form stable Dean vortical structures is determined at a Reynolds number in the range of 100 to 500. For purposes herein, a stable Dean vortical structure is present when a secondary flow or secondary motion is present and/or indicated in the flow, which for purposes herein also includes the representation of the flow e.g., when demonstrated via modeling and/or computer simulation, as is readily understood in the art. For purposes herein, the Reynolds number is determined according to the equation:

$$Re = \frac{uL}{v} = \frac{ρ uL}{μ}$$

wherein:

Re is the Reynolds number;

ρ is the density of the fluid;

u is the flow speed;

L is a characteristic linear dimension (of the flow channel);

μ is the dynamic viscosity of the fluid; and

ν is the kinematic viscosity of the fluid.

For purposes herein a sorbent refers to a substance which has the property of collecting and/or retaining molecules of another substance. This may be accomplished by sorption, including adsorption, absorption, sequestration, trapping and/or the like. This may also be accomplished by the occurrence of reversible or non-reversable chemical reactions, and/or combinations thereof. For purposes herein, sorbents also include multipurpose materials that utilize any number of processes to remove the target analyte from the fluid being treated. Sorbents may be solids, liquids and/or gels under the conditions at which they are utilized. Sorbents may also undergo phase transitions as a result of removing the target analyte from the fluid being treated and/or as a result of releasing the target analyte or a material derived therefrom. For purposes herein a sorbent present in a liquid phase refers to substances which readily flow under the force of gravity, having a viscosity of less than or equal to about 10,000 cps, preferably less than or equal to about 5000 cps, with less than or equal to about 1000 cps or less than or equal to about 100 cps being more preferred.

For purposes herein a sorbent may also be a catalyst depending on the intended use of the substrate. While a catalyst may not generally be considered a sorbent, for purposes herein it is to be understood that unless expressly stated otherwise, a sorbent may also refer to a catalyst even though the catalyst does not retain a target analyte, but instead facilitates a reaction to convert that target analyte to something else, e.g., for purposes herein a substrate that comprises a sorbent includes a substrate comprising a catalyst present in or on the substrate which converts CO₂ into a hydrocarbon. In this example, the “sorbent” is the catalyst.

For purposes herein, a thickness of a flow channel sidewall (or wall) is defined as the distance between an inner side of a first flow channel and an inner side of a directly adjacent flow channel such that the flow channel sidewall is the barrier between the two adjacent flow channels.

As used herein, Sherwood number (Sh), which is also referred to in the art as the mass transfer Nusselt number, is a dimensionless number used in mass-transfer operation. It represents the ratio of the convective mass transfer to the rate of diffusive mass transport and is defined as

$$S h = \frac{h}{D \text{/} L} = \frac{Convective mass transer rate}{Diffusion rate}$$

follows:

where

L is a characteristic length (m);

D is mass diffusivity (m^{2*}s⁻¹); and h is the convective mass transfer film coefficient (m*s⁻¹).

In particular for purposes herein, the Sherwood number is defined as a function of the Reynolds and Schmidt numbers depending on the operation, including the ratio of the mass transfer to frictional loses of a system at varying Reynolds number in which the Friction coefficient: C_{f}, is multiplied by the flow Reynolds number Re, according to the relationship Sₕ/C_{f}Rₑ.

In one embodiment, a capture device substrate comprises a fluid inlet in fluid communication with a fluid outlet through at least one flow channel disposed along at least one flow path disposed within a body; the flow channel comprising a cross-sectional shape comprising a plurality of sides defining a cross-sectional area, determined orthogonal to the flow path; at least a portion of the flow path comprising an essentially sinusoidal shape, an essentially helical shape, or a combination thereof, configured to produce one or more stable Dean vortical structures (Dean-like vortical structures, essentially Dean vortical structures, and/or vortical structures having a secondary flow in the base flow flowing through the flow channel) in a fluid flowing through the flow channel when determined at a Reynolds number from about 100 to 500; and a sorbent effective to absorb, adsorb, sequester, and/or undergo a chemical reaction with one or more components present in the fluid flowing through at least a portion of the flow channel.

In some embodiments, the capture device substrate comprises a first flow channel disposed proximate to a second flow channel, wherein at least a portion of at least one side of the first flow channel forms at least one common sidewall between at least a portion of at least one side of the second flow channel. In some of these embodiments, at least a portion of the at least one common sidewall comprises a porosity, a conduit, a via, or a combination thereof, wherein the fluid inlet is in fluid communication with the fluid outlet through at least a portion of the at least one common sidewall.

In some embodiments, the first flow channel is open on an inlet end of the body in direct fluid communication with the fluid inlet and closed on an outlet end of the body (i.e., an inlet channel), and the second flow channel is closed on the inlet end of the body and open on an outlet end of the body in direct fluid communication with the fluid outlet (i.e., an outlet channel).

In embodiments, at least a portion of the flow path (the shape of the flow channel) comprises an essentially sinusoidal shape comprising an amplitude and a wavelength configured to produce the stable Dean vortical structures in the fluid flowing through at least a portion of the flow channel.

In some embodiments at least a portion of the flow path comprises an essentially helical shape oriented radially about a center axis of the flow channel and comprising a radius and a pitch configured to produce the stable Dean vortical structures in a fluid flowing through at least a portion of the flow channel.

In some embodiments, at least a portion of the flow path comprises an essentially helical shape radially arranged about an essentially sinusoidal shape and comprising an amplitude, a wavelength, a radius and a pitch configured to produce the stable Dean vortical structures in the fluid flowing through at least a portion of the flow channel. In other embodiments, at least a portion of the flow path comprises an essentially sinusoidal shape arranged within an essentially helical shape oriented radially about a center axis of the flow channel, comprising an amplitude, a wavelength, a radius and a pitch configured to produce the stable Dean vortical structures in the fluid flowing through at least a portion of the flow channel.

In embodiments, at least a portion of the body comprises a plurality of flow channels, at least a portion of the plurality of flow channels comprising a flow path comprising an essentially helical shape coaxially disposed about a single axis of the plurality of flow channels, each of the plurality of flow channels comprising a flow channel centerline defined by a geometric center of the cross-sectional shape of the flow channel at each point along a length of the portion of the body, the flow paths of each of the plurality of flow channels dimensioned and arranged within the portion of the body such that each of the flow channel centerlines are essentially equal in length.

In embodiments, at least a portion of the body comprises a plurality of flow channels, at least a portion of the plurality of flow channels comprising a flow path comprising an essentially helical shape coaxially disposed about a center axis of the corresponding flow channel, wherein the cross-sectional area of the flow channel varies periodically between a minimum value and a maximum value when determined along the center axis of the flow channel.

In embodiments, at least one flow channel has a cross-sectional shape comprising 3 or more sides. In some embodiments, at least a portion of the substrate is formed from one or more ceramics, metals, sorbents, thermoplastic polymers, thermoset polymers, or a combination thereof.

In embodiments, the substrate comprises or is formed from one or more metal sheets, polymeric sheets, or a combination thereof, disposed about at least one axis of the body. In some of such embodiments, at least a portion of the substrate comprises or is formed from a plurality of corrugated sheets separated from one another by a corresponding number of flat sheets wherein contact between the corrugated sheet and the flat sheet forms the cross-sectional shape of the flow channels; a plurality of corrugated sheets having a first cross-sectional shape separated from one another by a corresponding number of corrugated sheets having a second cross-sectional shape, wherein contact between the corrugated sheets forms the cross-sectional shape of the flow channels; or a combination thereof.

In embodiments, the body of the substrate comprises an inlet end in fluid communication with the fluid inlet of the capture device, and an outlet end in fluid communication with the fluid outlet of the capture device, and wherein the cross-sectional area of each flow channel disposed within the body is essentially uniform from the inlet end to the outlet end of the body.

### Sorbents

In embodiments, the direct capture substrate further comprises one or more sorbents. As used herein, a sorbent is effective to absorb, adsorb, sequester, and/or undergo a chemical reaction with a target compound in the fluid being treated. In one embodiment, the target compound is carbon dioxide.

Suitable sorbents include, but are not limited to oligomeric amines e.g., polyethyleneimine (PEI), and tetraethylenepentamine, (TEPA), functionalized mesoporous silica capsules, e.g., MC400/10 nano capsules, zeolites, (e.g., 5A, 13X, NaY, NaY-10, H-Y-5, H-Y-30, H-Y-80, HiSiv 1000, H-ZSM-5-30, H-ZSM-5-50, H-ZSM-5-80, H-ZSM-5-280, and HiSiv 3000, and the like, hierarchical silica monoliths, mesoporous silica SBA-15 (SBA(P)) with tetraethylenepentamine (TEPA) and/or polyethyleneimine (PEI), carbon nanotubes, metal-organic frameworks, M₂(dobpdc) (M=Zn (1), Mg (2); dobpdc⁴⁻=4,4′-dioxido-3,3′-biphenyldicarboxylate), adopting an expanded MOF-74 structure types, amine-grafted silicas, aqueous amine solutions, polyamines in porous polymer networks, pore-expanded silica (e.g., MCM-41) with diethanolamine and/or 3-[2-(2-aminoethylamino) ethylamino]propyl trimethoxysilane (TRI) and/or the like, high-silica zeolites TNU-9, IM-5, SSZ-74, ferrierite, ZSM-5 and/or ZSM-11, Y-type zeolite with a Si/A1 molar ratio of 60 (abbreviated as Y60) modified with amines including PEI and TEPA, mesoporous silica (e.g., SBA-15) modified with 3-trimethoxysilylpropyl diethylenetriamine, beta zeolites, activated carbon, activate carbon with ammonia or other amines, mesoporous silica foam comprising tethered amines, hollow fibers comprising amine impregnated silica, aqueous amines e.g., mono, di and tri alkyl amines and mono, di and tri alkanol amines e.g., monoethanolamine (MEA), activate carbon with carbonates e.g., potassium carbonate, sorb NX35, olivine, modified alumina with KAl(CO₃)(OH)₂, combinations thereof, and the like.

In embodiments, the sorbent is disposed on or at least partially within walls of the flow channels. In some embodiments, the substrate is at least partially constructed from the sorbent and/or the substrate is functionalized with the sorbent. In some embodiments, the sorbent is present in a liquid, gel and/or slurry mobile phase flowing through one or more of the plurality of channels, which in an embodiment may be a counter-current flow to the fluid to be treated flowing therethrough. In some embodiments, the mobile phase flowing sorbent is directed into the one or more flow channels through one or more channels laterally disposed into the body at an angle to the flow path of the flow channel.

In embodiments, a method to remove a target compound from a fluid comprises the steps of directing the fluid comprising a first concentration of the target compound through a capture device comprising a capture device substrate according to one or more embodiments disclosed herein at a flow rate, a temperature, and for a period of time sufficient to produce a treated stream having a second concentration of the target compound, wherein the first concentration of the target compound is greater than the second concentration of the target compound. In some embodiments, the method further comprises a desorption step wherein the capture device substrate is subjected to conditions suitable to release the target compound.

In embodiments, the fluid to be treated is air and the target compound includes carbon dioxide.

In embodiment, the sorbent is disposed on or at least partially within the flow channels of the substrate. Suitable methods include various coating procedures wherein the sorbent is used alone or in combination with a support material e.g., mesoporous alumina, silica, and/or the like. The sorbent such as PEI, used as a viscous liquid or in solvent is directed through the flow channels as a slurry or a solution depending on the sorbent used. Various solvents and binders may be employed and the solvent are then removed.

In other embodiments, the direct capture substrate is functionalize using wet impregnation wherein the sorbent is combined with a solvent and optionally a support which is directed through the flow channels. The solvent is then evaporated. This can also be done without solvent.

In other embodiments, the substrate is produced via binder jetting or other similar technologies to form a porous substrate which is then sintered. The sintered substrate is then functionalized with the sorbent, typically by combining the sorbent with solvent and directing the sorbent through the channels, e.g., immersing the substrate in the sorbent mixture with agitation. After which the solvent is evaporated. This may be repeated over again using the same or a different sorbent.

Accordingly, in embodiments the sorbent is disposed on or within the flow channel walls using wash coating, incipient wetness, impregnation, and variations thereof known in the art. In another embodiment, the substrate is composed of support material such as mesoporous silica or mesoporous alumina and then functionalized with a sorbent material, such as polyethyleneimine (PEI), via wet impregnation or some other method. This allows for a reduction in the thermal mass of the contactor relative to a baseline contactor that is composed of an inert material, such as cordierite, and then coated with a sorbent/support material.

In another embodiment, the contactor is composed entirely of sorbent material, and/or sorbent material disposed on a support such as PEI on silica/alumina. This may allow for a further reduction in thermal mass.

### Capture Device Substrates

In one embodiment, a capture device comprises a capture device substrate, also referred to herein as a honeycomb. The capture device substrate may be monolithic or may comprise a plurality of substrates. The capture device substrate may have a plurality of flow channels, each having essentially the same shaped flow path from an inlet to the outlet, or may have a plurality of flow channels having a plurality shapes of the individual flow paths. This plurality of shapes of the individual flow paths may be consistent from the inlet to the outlet of the substrate, e.g., a substrate having a plurality of essentially sinusoidal flow channels running from the inlet to the outlet of the substrate disposed within a plurality of essentially helical flow channels running from the inlet to the outlet of the substrate; and/or in other embodiments, the plurality of shapes of the individual flow paths may be arranged within the substrate body in various sections of the substrate from the inlet to the outlet of the substrate, e.g., a substrate having a plurality of essentially sinusoidal flow channels present in a first portion of the substrate (the inlet of the first portion to the outlet of the first portion) followed by a second portion having a plurality of essentially helical flow channels running from the inlet of the second portion to the outlet of the second portion. The various portions may be oriented perpendicular to the overall flow path through the capture device, may be parallel to the overall flow path through the capture device, or may be oriented at various angles to the overall flow path through the capture device.

Each of the flow channels may individually have a single inlet and a single outlet, multiple inlets and multiple outlets, a single inlet and multiple outlets, or multiple inlets and a single outlet. The number of flow channels and/or the average flow channel cross-sectional area present at a particular point in a cross section of the capture device substrate may be variable along the length of the capture device substrate or substrates, e.g., the capture device may have a substrate comprising a first number of channels per unit area present at a point proximate to the inlet of the capture device which is different from a second number of channels per unit area present in the substrate located at a point proximate to the outlet of the capture device, and/or the substrate present at a point proximate to the inlet of the capture device may have channels having a first cross-sectional area which is different from a second cross-sectional area of the same channels located at a point proximate to the outlet of the capture device.

Applicant has discovered that the capture device substrates disclosed herein, when compared to capture device substrates having linear flow channels as seen in prior art FIGS. 2 and 3, yield at least twice as much mass transfer, i.e., throughput, and/or Sherwood number, which is defined as a dimensionless number used in mass-transfer operation representing the ratio of the convective mass transfer to the rate of diffusive mass transport. Accordingly, capture devices according to the instant disclosure allow for downsizing, reduced sorbent, and/or much improved yield.

Applicant has discovered that when employing the capture device substrates according to embodiments disclosed herein, the mass transfer increases faster than its frictional losses. As a result, the presently claimed invention yields a net gain in Sₕ/C_{f}.Re; that is, its required pumping power is reduced by downsizing, while still meeting its performance target. Additionally, it requires less energy for desorption due to the reduced capture device substrate thermal mass. Applicant has also discovered that a capture device substrate or honeycomb made of metal, a thermoplastic, a thermoset plastic, and/or a combination thereof, instead of ceramic or other non-conductive materials, permits efficient heating strategies such as joule heating, in lieu of the less efficient steam heating required by ceramic honeycombs, thus providing increased energy cost savings during a desorption operations, in addition to having a reduced thermal mass allowing for much faster return to sorbet operation than devices known in the art. In addition, applicant has discovered that capture device substrates according to embodiments disclosed herein can be manufactured out of thermoplastic and/or thermoset polymers e.g., alpha olefin, acrylics, polyesters, polyethers, polyimines, polyamides, and/or the like, and thus may be produced at greatly reduced cost relative to substrates known in the art. Applicant has further discovered that the capture device substrates may be produced at least partially from sorbents, e.g., PEI, and/or may be produced by additive manufacturing techniques, that simply production and reduce cost. Suitable polymers, generally referred to herein as “plastics” include polyethylene, isotactic polypropylene, highly isotactic polypropylene, syndiotactic polypropylene, random copolymer of propylene and ethylene, and/or butene, and/or hexene, polybutene, ethylene vinyl acetate, LDPE, LLDPE, HDPE, ethylene vinyl acetate, ethylene methyl acrylate, copolymers of acrylic acid, polymethylmethacrylate or any other polymers polymerizable by a high-pressure free radical process, polyvinylchloride, polybutene-1, isotactic polybutene, ABS resins, ethylene-propylene rubber (EPR), vulcanized EPR, EPDM, block copolymer, styrenic block copolymers, polyamides, polycarbonates, PET resins, cross linked polyethylene, copolymers of ethylene and vinyl alcohol (EVOH), polymers of aromatic monomers such as polystyrene, poly-1 esters, polyacetal, polyvinylidine fluoride, polyethylene glycols, polyisobutylene, and/or combinations thereof.

As shown in FIG. 7, a portion of a curved flow channel 700 which may be according to any one or more embodiments disclosed herein i.e., flow channels having an essentially helical shaped flow path, an essentially sinusoidal shaped flow path, an essentially helical-essentially sinusoidal shaped flow path, and/or an essentially sinusoidal-essentially helical shaped flow path, when operated within a laminar flow range according to embodiments disclosed herein, have been discovered both computationally 702 and experimentally 704 to form a secondary flow known as Dean flow or Dean vortex (Dean vortical structures), in which a pair of counter-rotating vortex structures enhance transport of energy (e.g., heat) and target species (e.g., mass) to and from the walls of the flow channel. Dean vortices are known to be good mixers. The change in the flow and the secondary flows of the example shown in FIG. 7 are shown in slices along the flow channel in FIG. 8. For purposes herein while these Dean Vortices may form at a Reynolds number of 0.5 or more up to, and possibly beyond turbulent flow, the presence of such Dean Vortices in a flow channel according to the instant disclosure is apparent at a Reynolds number from about 100 to 500, even though the Dean Vortices may form at substantially lower (e.g., at a Reynolds number from about 0.5 to 99) and/or substantially higher (e.g., at a Reynolds number from about 500 to greater than or equal to about 1000, or greater than or equal to about 1500, or greater than or equal to about 2,000, or more.

In the flow channels according to embodiments disclosed herein, transport phenomena is abundant due to the presence of Dean vortices in essentially helical and essentially sinusoidal geometries, which have been discovered to increase heat and mass transfer from about 200% up to an in excess of about 500% relative to linear flow channels, even in a low Reynolds number regime e.g., about 1 to 50.

In embodiments, mass transfer takes place in a convective regime such that transport improvements due to Dean vortices have the effect of transforming a diffusion-dominated regime present in a linear channel (see FIG. 2) to a convective one in the disclosed essentially helical channels, which greatly improves the utility of the instant capture device substrates in CO₂ capture (see FIGS. 7 & 8).

Likewise, these same Dean vortical structure flows improve the desorption of the sequestered components thus improving overall system throughput and efficiency.

Direct capture substrates comprising substrates in which at least a portion comprises a metallic and/or polymeric honeycomb (metallic and/or polymeric capture device substrates) offer a number of advantages against their ceramic counterparts. These honeycombs according to embodiments disclosed herein offer improved structural rigidity, wider flexibility and the ability to select thinner walls to achieve a reduced thermal mass compared to ceramic capture device substrates.

The increased thermal conductivity of the metallic capture device substrates is ˜14 times greater than that of a ceramic and provides a more rapid and uniform heat dispersion throughout the honeycomb relative to a ceramic substrate. Moreover, unlike ceramic honeycombs, metallic ones may be heated by passing an electric current through the honeycomb itself, with this heating efficiency i.e., the power factor, being close to 100%, which is unobtainable by steam heating currently used in ceramic of other systems. In addition, applicant has discovered a process to manufacture the complex essentially helical channels which is an improvement over methods known in the art for producing linear channel substrates.

Consistent with the American Physical Society CO₂ cost model, applicant has discovered improvements in pumping power, capture efficiency during adsorption, correlations for pumping power, and mass transfer when essentially helical channels according to embodiments disclosed herein are utilized.

As the flow travels along the essentially helical path of the channel, counter-rotating Dean vortex structures are formed enhancing the rate of mass transfer of CO₂ to/from the sorbent per unit area (also known as mass flux), characterized by an increase in Sherwood number (see FIG. 20). A higher Sherwood number allows for the same capture efficiency (percentage of CO₂ captured) in a reduced honeycomb volume, aka downsizing. An increase in the flow rate offers even further downsizing of the honeycomb, ranging from a reduction by 40% at relatively low channel velocities (about 1 m/s or a Reynolds number of 75) to 80% at higher channel velocities (about 14 m/s or a Reynolds number of 1,000).

While Dean vortices created in the essentially helical channels increase the rate of mass transfer to/from the sorbent, they also increase the flow-wall friction, which is characterized by the coefficient of friction C_{f} and the Reynolds number Re and hence, the pressure drop. As is readily known to one of skill in the art, the higher the pressure drop the more pumping power required to force the flow through the channels. However, as shown in FIG. 21, Applicant discovered that utilizing embodiments of the capture device substrate disclosed herein, as the flow rate increases the Sherwood number increases more rapidly than the friction factor, allowing for increased capture efficiency (increased percentage of CO₂ captured) for the same pressure drop or pumping power. Accordingly, the essentially helical channels according to embodiments disclosed herein are useful in reducing the required pumping power and by extension, the energy cost, for a given amount of captured CO₂ relative to sorbent devices having linear flow channels.

In embodiments, direct air capture of CO₂ involves a sorbing step in which ambient air or a fluid from another source is directed through the capture device, during which the CO₂ is captured by the sorbent material. In some embodiments, a second step include heating of the capture device substrate, and/or reducing the pressure on the substrate, and/or applying an electric potential or switching the polarity of the electric potential, and/or other conditions which cause the sorbent to release the CO₂, which is directed into a storage facility or otherwise processed for storage or use.

The bulk of the energy consumed in DAC is due to the thermal energy required for desorption. This energy can be divided into three parts. First is the energy required to heat the honeycomb, second the energy required to heat the sorbent, and third, the energy necessary to break the chemical bonds between the sorbent and CO₂. The latter two vary with the sorbent type, while the first depends on the honeycomb volume and its thermophysical properties such as density and specific heat of the substrate material. There exists a significant difference in the amount of energy required for heating a ceramic vs. a metallic and/or polymeric honeycomb to trigger and to sustain desorption. As shown in Table 1 below, embodiments according to the instant disclosure provide for significant energy savings when a metallic essentially helical channel honeycomb having thin walls is utilized, yielding greater than about 10%, or 20% or 30% energy saving.

TABLE 1
Comparison of energy budgeting for standard vs. metallic
essentially helical honeycombs per each metric ton of CO₂ captured.

| | Pumping | | | |
|---|---|---|---|---|
| | Energy | Thermal | | |
| | during | Energy | TOTAL | |
| | absorption | of desorption | ENERGY | |
| | (kWh/tonne) | (kWh/tonne) | (kWh/tonne) | Result |
| Standard linear direct | 160 | 1675 | 1835 | Reduce |
| capture device | | | | substrate |
| Inventive direct | 110 | 1230 | 1340 | volume |
| capture device with | | | | (thermal mass) |
| essentially helical | | | | by 40-80% |
| metallic substrate | | | | (depends on |
| Difference | Essentially | Essentially | Essentially | flow rate) |
| | helical is 31% | helical is 27% | helical is 27% | |
| | lower | lower | lower | |

The use of metallic direct capture substrates according to embodiments disclosed herein further enables their heating via electricity or induction, thus avoiding energy losses/inefficiencies in using steam or heated gas via convective heating.

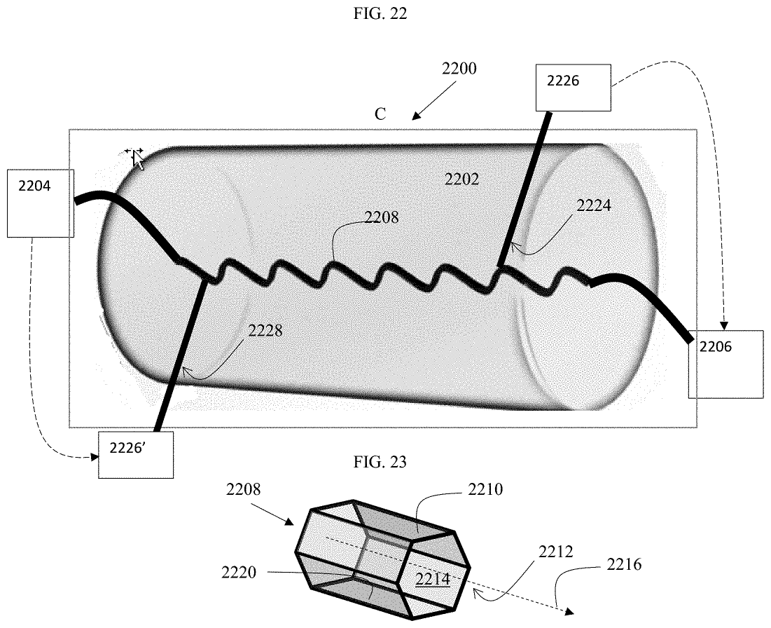

As shown in FIG. 22, in one or more alternative embodiments, a capture device, generally referred to as 2200 comprises a fluid inlet 2204 in fluid communication with a fluid outlet 2206 through at least one flow channel disposed along at least one flow path disposed within a body; the flow channel 2208, a portion of which is shown in FIG. 23, comprising a cross-sectional shape 2212 comprising a plurality of sides 2210 defining a cross-sectional area 2214, determined orthogonal to the flow path 2216; at least a portion of the flow path comprising an essentially sinusoidal shape, an essentially helical shape, or a combination thereof, configured to produce one or more stable Dean vortical structures in a fluid flowing through the flow channel when determined at a Reynolds number from about 100 to 500; and a sorbent 2220 effective to absorb, adsorb, sequester, and/or undergo a chemical reaction with one or more components present in the fluid flowing through at least a portion of the flow channel. As shown in FIG. 22, the capture device substrate 2202 further includes one or more liquid sorbent inlets 2224 in fluid communication with flow channel 2208 and a liquid sorbent reservoir 2226 and one or more liquid sorbent outlets 2228 a fluid communication with flow channel 2208 and a liquid sorbent reservoir 2226′, which may be the same or different as the reservoir on the sorbent inlet channel. As indicated by the dashed lines, the liquid sorbent inlet 2224 may be directed through the fluid outlet 2206 and the liquid sorbent outlet 2228 may directed through the fluid inlet 2204 of the direct capture device.

As the fluid having the target compounds (e.g., air comprising CO₂) flows through the substrate flow channels, the CO₂ encounters a liquid sorbent flowing through the flow channels, preferably the liquid sorbent is flowing counter-currently to the main fluid, wherein the target material is adsorbed and is later separated. In embodiments, the capture device substrates are configured to allow CO₂-laden air to come in contact with liquid sorbent which preferably flows through the substrate via gravity and is collected for desorbing or other processing. In some embodiments, the liquid sorbent is directed into the outlet of the flow channels. In other embodiments, the liquid sorbent is directed into and optionally out of the one or more flow channels through one or more auxiliary channels laterally disposed into the body at an angle to the center axis of the body, typically from about 90° to about 10° with respect to the center axis of the substrate. In some embodiments, these lateral channels may intersect a particular flow channel at a number of points along the length of the flow channel. In other embodiments, the liquid sorbent may enter into the flow channels via neighboring auxiliary channels longitudinally disposed into the capture device substrate for this purpose, which are in fluid communication with one or more adjacent flow channels at one or more points along the length of the flow channel.

As shown in FIG. 19A, capture devices 1902 comprise a capture device substrate 1912 according to the instant disclosure, which comprise an inlet end 1906 separated from an outlet end 1904 by a body length 1918, wherein the inlet end 1906 is in fluid communication with the outlet end 1904 through a plurality of channels disposed therethrough. In some embodiments, as shown in FIGS. 4 and 5, the channels have an essentially helical shape 25, disposed through the substrate body about a center axis 30 of the helix. Each of the channels comprise a cross-sectional shape 27 which is determined orthogonal to the helix centerline 30 having a plurality of sides 28, and a channel centerline 29 defined by a geometric center of a cross-section of the particular channel at each point along the body length or a portion of the body length from the inlet end of the substrate or portion of the substrate body to the outlet end of the substrate or portion of the substrate body.

As shown in FIG. 19B, in an embodiment, the capture device substrate 1912 may comprise a plurality of substrate portions 1912a, 1912b, and 1912c arranged perpendicular to the flow therethrough. As shown in FIG. 19C, in an embodiment, the capture device substrate 1912 may comprise a plurality of substrate portions 1912a, 1912b, and 1912c arranged parallel to the flow therethrough.

### Essentially Helical Flow Channels

In one or more embodiments of the substrate, each of the essentially helical channels comprise a radius R equal to a distance determined orthogonal to the central axis from the channel centerline to the central axis of a channel, and a pitch P equal to a length of the channel centerline 29 through one complete rotation of the channel about the central axis 30 according to the equation P=2πK such that the body length H=PN=2πKN, wherein N is the number of rotations of the channel about the central axis from the inlet end to the outlet end, the length of the channel centerline L is according to the equation:

L=2πN\sqrt{R²+K²};

a ratio of the length of the channel centerline L to the body length H is defined by the equation:

$$\frac{L}{H} = \frac{2 π N \sqrt{{R}^{2} + {K}^{2}}}{2 π N K} = \frac{\sqrt{{R}^{2} + {K}^{2}}}{K} .$$

In each embodiment, the channels have a cross-sectional shape comprising 3 or more sides and, in some embodiments, up to an infinite number of sides. The cross-sectional shape may be regular or irregular, may comprise a plurality of essentially linear sides, smooth curved sides, essentially sinusoidal or wavy sides, or any combination thereof. In all embodiments, the channels are dimensioned such that a fluid flowing from the inlet end to the outlet end at a flow rate consistent with an intended use of the substrate forms a plurality of secondary flows having a Dean vortex-type flow pattern (see FIG. 7) within one or more of the channels.

Treatment of fluids utilizing capture device substrates which involve catalytic reactions between target species in the fluid and a catalyst disposed on or within the channel walls typically require longer residence times. The efficiency of a capture device substrate can be improved by increasing the residence time of the fluid within the substrate, by increasing interactions between the fluid flow and the channel walls of the substrate, and the like. The standard arrangement of channels within capture device substrates common in the art involves linear channels. The fluid flow through these channels is normally laminar at slow and moderate gas flow rates typical of direct air capture and/or exhaust gas treatment, and/or the like. The catalytic reaction efficiencies within linear catalytic channels are rate-limited by the length of the channel and amount of catalyst substrate within channels at constant flow rates. However, as the length of the substrate increases, and/or as the size of the individual channels decrease, the backpressure or resistance to flow caused by the substrate increases. This increase in backpressure requires more energy and thus lowers the overall efficiency of a system employing such a capture device substrate.

Applicant has unexpectedly discovered, however, that non-linear channel geometry results in a dramatic increase in catalytic and other efficiencies of systems employing capture device substrates according to the instant disclosure.

As shown in FIG. 2, representative of the prior art, a prior art direct capture substrate 5 comprises linear flow channels 21, only a single linear channel is shown for clarity. The fluid flows through an inlet opening 9 and travels through the channel 21 in an essentially laminar flow and exits the channel 21 through an outlet opening 10. FIG. 4 shows is representation of a single channel having a non-linear flow path with an essentially helical shape, generally indicated as 11. As shown in FIG. 4, as the fluid 16 flows through the essentially helical channel 11, the laminar flow is interrupted resulting in the formation of secondary flow paths which depending on the shape of the channel. The curvature 17 of the essentially helical channel 11 results in internal flow transitions into eddies and Dean vortical structures therein, resulting in the formation of strong secondary flows which induce centrifugal forces within the flow. However, the formation of these secondary flow paths is known in the art to increase the backpressure or resistance to flow through the channel. Applicant has discovered that the by controlling the shape of the flow channel, secondary flow paths of the Dean vortices and/or having Dean vortical-like flow patterns, form within the fluid flowing through the essentially helical flow channel. While FIG. 4 shows a right-handed helix, applicant has further discovered that these same Dean vortices can be made to occur in left-handed helices as well.

Applicant has discovered that as the fluid (a gas) flows through the essentially helical flow channel, the forces exerted by and on the fluid flow by the flow through the essentially helical flow channel effect the flow in a complex way in which the gas is compressed and expanded.

As noted above, a useful measure of the effect of the channel shape on the fluid flow includes the Reynolds number (Re) which is a dimensionless quantity indicative of flow patterns in different fluid flow situations. However, the more specialized Dean number has also been found suitable for characterizing flow through capture device substrates according to the instant disclosure. For purposes herein, the Dean number (De) is a dimensionless group which occurs in the study of flow in curved pipes and channels. The Dean number is typically denoted by De (or Dn). For a flow in a pipe or tube it is defined as:

$$De = \frac{\sqrt{\frac{1}{2} ( inertial forces ) ( centripetal forces )}}{viscous forces} = \frac{\sqrt{\frac{1}{2} ( ρ {D}^{2} {R}_{c} \frac{{v}^{2}}{D} ) ( ρ {D}^{2} {R}_{c} \frac{{v}^{2}}{{R}_{c}} )}}{μ \frac{v}{D} {DR}_{c}} = \frac{ρ Dv}{μ} \sqrt{\frac{D}{2 {R}_{c}}} = Re \sqrt{\frac{D}{2 {R}_{c}}}$$

wherein ρ is the density of the fluid;

μ is the dynamic viscosity;

v is the axial velocity scale;

D is the diameter (for non-circular geometry, an equivalent diameter is used;

R_{c} is the radius of curvature of the path of the channel, and

Re is the Reynolds number.

Accordingly, the Dean number is the product of the Reynolds number (based on axial flow v through a pipe of diameter D and the square root of the curvature ratio. As is readily understood, low Dean numbers (De<40˜60) represent unidirectional flows. As the Dean number increases e.g., 64˜75, wavy perturbations are observed in the cross-section indicating secondary flow. At higher Dean numbers e.g., greater than ˜75, a pair of Dean vortices become stable, indicating a primary dynamic instability. A secondary instability appears for De>75˜200, where the vortices present undulations, twisting, and eventually merging and pair splitting. Fully turbulent flow forms at De>400. Applicant has further discovered that the flow rates and the mixing or chaotic intensities of the Dean vortices (i.e., the Dean number De) may, among other things, depend on the pitch of the helix 19 (one complete turn) and the diameter of the helix 20.

### Essentially Sinusoidal Channels

Another embodiment of a non-linear catalyst substrate is a flow channel having a flow path comprising an essentially sinusoidal shape, as shown in FIG. 6, which shows a single essentially sinusoidal shaped flow channel 22 dispersed through the substrate body. The fluid enters through an inlet 9 and flows through the essentially sinusoidal channel 22, wherein eddies and vortexes are formed due to the shape and curvature of the channel. Applicant has discovered that the nature of flow through the essentially sinusoidal channel results in unique flow patterns involving Dean vortices and Dean vortex-like flows which are different from both linear and essentially helical flow channels. Applicant has further discovered that the flow patterns which occur with the fluid flowing through the essentially sinusoidal channels may be controlled and optimized for certain outcomes by selection of the wave length 24 and the amplitude 26 of the essentially sinusoidal shape of the flow channel, as determined along a centerline of the flow channel. By varying the wavelength 24 and the amplitude 26 of the essentially sinusoidal flow channel, applicant has achieved marked variations in flow rates, backpressure, formation of eddies, and mass transport of analytes to the channel walls as the fluid flows through the essentially sinusoidal channels.

In embodiments, the essentially helical channels and/or essentially sinusoidal channels are dimensioned and arranged according to embodiments disclosed herein, Dean vortices and the like provide a secondary flow lateral to the base flow, enhancing the flux of flow species toward the channel walls, allowing increased sorbent actions.

In embodiments, the flow cross section of the channels according to one or more embodiments may be varied to alter the cross sectional shape and efficiency of a flow channel for a particular purpose. That includes, but is not limited to, designing other types of flow cross sections. The cross-sectional shape of flow channels must comprise at least 3 sides, i.e., have generally triangular cross-sections shape determined orthogonal to the center axis of the flow channel. In other embodiments, the cross-sectional shape of flow channels may comprise at least 4 sides, or at least 5 sides, or at least 6 sides, or at least 7 sides, or at least 8 sides, or may comprise an infinite number of sides, i.e., be circular, oval, or the like. In some embodiments, the number of sides of the flow channels also changes and/or the cross-sectional shape of the flow channel is variable along the body length.

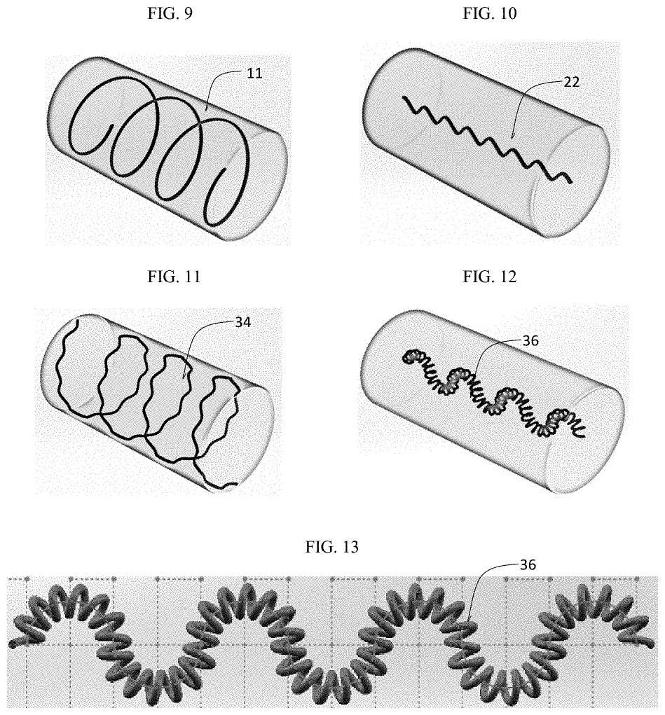

In some embodiments, each of the sides of the cross-sectional shape of the flow channels are essentially equal, in other embodiments, at least two of the sides of the cross-sectional shape of the flow channels are different. In embodiments in which the cross-sectional shape of the flow channels have an infinite number of sides, the sides may be uniformly radially disposed about a center point, i.e., circular cross-section, or may be non-uniformly centered about the center point, e.g., having an oval shaped cross-section. While not a limiting factor in at least some embodiments, the capture device substrates according to embodiments disclosed herein may have from 1 to about 1000 or more flow channels per square inch of inlet surface. However, for the sake of illustrative simplicity, only one channel is illustrated in the figures where indicated for clarity. FIG. 9 shows a treatment device according to one or more embodiments disclosed herein, comprising a capture device substrate comprising an essentially helical channel 11. FIG. 10 shows a treatment device according to one or more embodiments disclosed herein, comprising a capture device substrate comprising an essentially sinusoidal channel 22. FIG. 11 shows a treatment device according to one or more embodiments disclosed herein, comprising a capture device substrate comprising an essentially helical-essentially sinusoidal channel 34 wherein an essentially sinusoidal shape is superimposed on a primarily essentially helical channel meaning the essentially helical shape is disposed along the length of the substrate. FIG. 12 shows a treatment device according to one or more embodiments disclosed herein, comprising a capture device substrate comprising an essentially sinusoidal-essentially helical channel 36 wherein an essentially helical shape is superimposed on a primarily essentially sinusoidal channel disposed along the length of the substrate. A side view of the essentially sinusoidal-essentially helical channel is shown in FIG. 13.

In some embodiments the flow channels are arranged within the substrate body such that a center axis of symmetry of each of the flow channels are parallel to one another, and which may also be parallel to the center axis of the body. In other embodiments, the flow channels are arranged radially about an axis of the body, which in some embodiments may be the center axis of the body. In still other embodiments, the channels are arranged in a nested fashion. In some nested embodiments, the channels are arranged such that each channel is separated from the next by a common channel wall, having a first side interior to a first channel and a second side interior to a second channel.

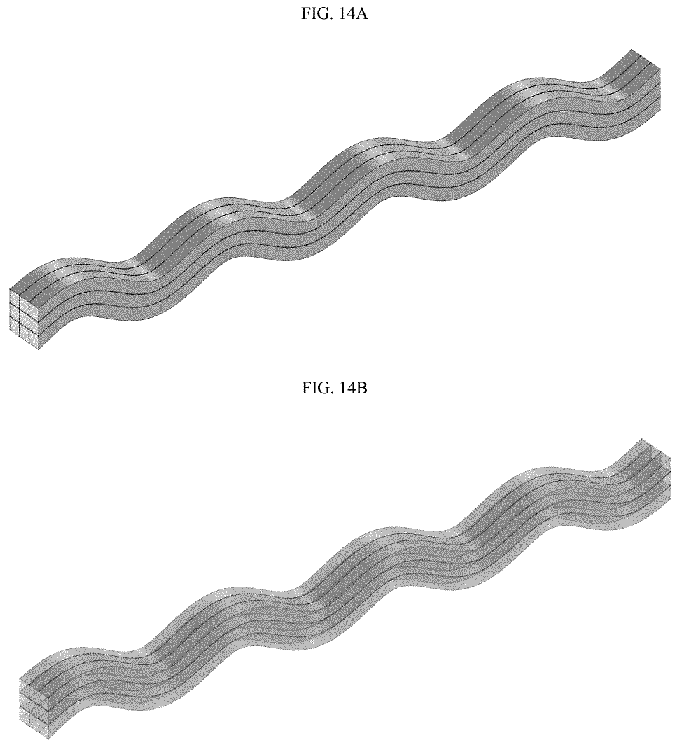

FIG. 14A shows a solid view and FIG. 14B shows a partially transparent view of a plurality of essentially sinusoidal channels having a square cross-sectional shape (4 sides) according to embodiments disclosed herein. In the embodiments shown, each channel has at least two sides in common with a neighboring channel. In some embodiments as shown in FIG. 14B, the thickness of the channel walls are uniform throughout.

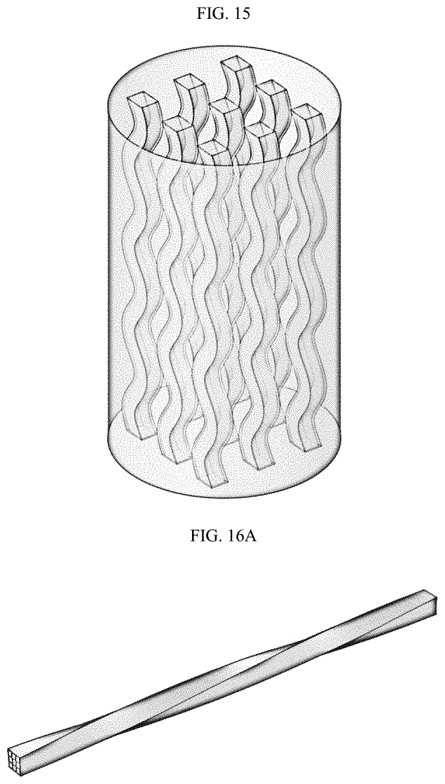

FIG. 15 shows a plurality of essentially sinusoidal channels having a square cross-sectional shape disposed within a substrate body according to embodiments disclosed herein.

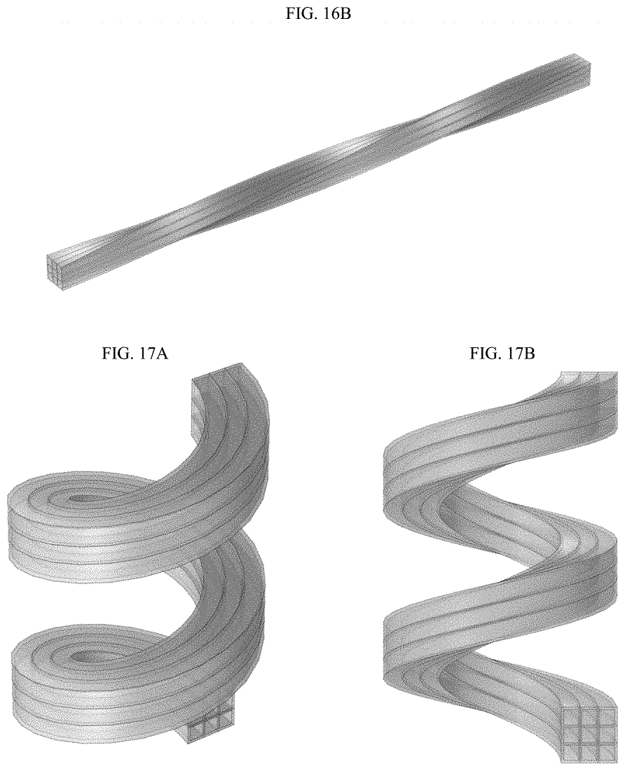

FIG. 16A shows a solid view and FIG. 16B shows a partially transparent view of a plurality of essentially helical flow channels having a square cross-sectional area. FIGS. 17A and 17B show views of alternative essentially helical flow channels having a much shorter pitch than those shown in FIG. 16B. In these embodiments, the essentially helical channels are arranged such that each flow channel has at least two sides in common with an adjacent or neighboring flow channel.

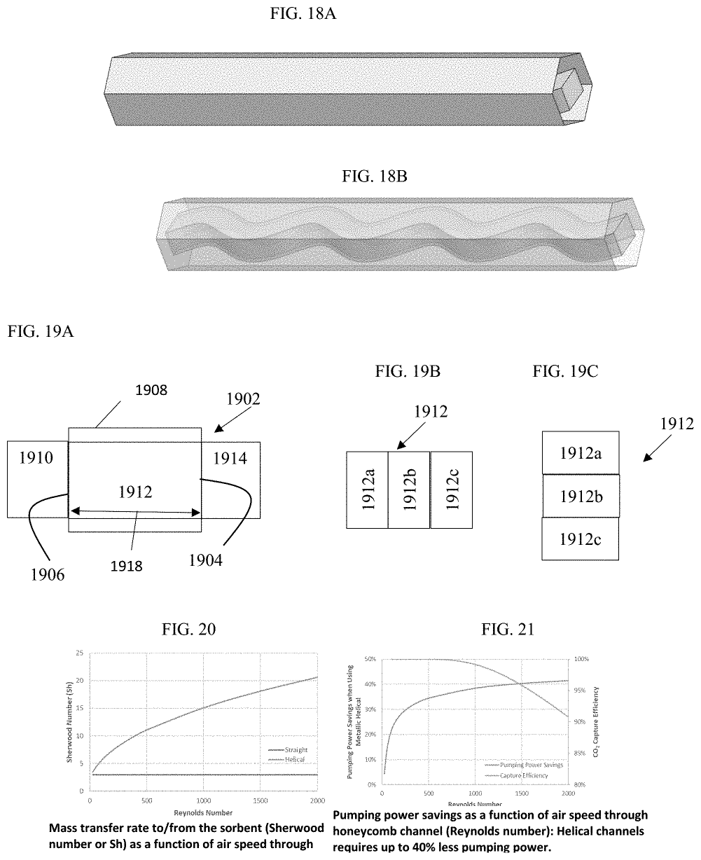

FIG. 18A shows a flow channel having an essentially sinusoidal flow path disposed within a hexagonal flow path in which the essentially sinusoidal flow path is produced around the inner essentially sinusoidal flow path.

The essentially helical and/or essentially sinusoidal flow channels according to embodiments disclosed herein independently form secondary flow vortices within the fluid flowing therethrough. When the two essentially helical and essentially sinusoidal channel types are combined (i.e., a flow channel having a flow path shape defined by two or more essentially sinusoidal channels superimposed on one another, two or more essentially helical channels superimposed on one another, an essentially helical-essentially sinusoidal and/or an essentially sinusoidal-essentially helical flow path shape) the resultant structure forms cumulatively stronger secondary vortices compared to either an essentially helical channel or a essentially sinusoidal channel alone. In all embodiments, the formation of Dean vortices and the pattern of secondary flow continually carries the fluid toward and in contact with the channel walls e.g., in contact with the catalyst coated channel walls, where heat, mass transfer, adsorption, absorption, desorption, chemical reactions, filtration, oxidation, and/or the like take place to treat the fluid flowing therethrough. Accordingly, the shape of the flow channel flow path according to one or more embodiments disclosed herein results in an overall improvement in sorption, catalytic and/or other treatment efficiency. In embodiments, the improvement in sorption efficiencies for capture device substrates according to one or more embodiments disclosed herein is at least 2 time greater, or 4 times greater, or 10 time greater than a comparative capture device substrate with linear channels (i.e., having the same length, cross-sectional area, sorbent and sorbent loading) when determined under essentially the same conditions.

In embodiments, a capture device substrate comprises a plurality of flow channels, preferably a plurality of identically-sized flow channels formed along a longitudinal axis of symmetry of the capture device substrate body, wherein the flow channels have channel centerlines that are non-coincident with each other, and wherein each of the flow channels is configured into an essentially helical substrate having a selected essentially helical diameter, a selected channel length, and a selected winding number of essentially helical turns, independent of the channel length, and wherein the winding number is selected in order to optimize a pressure gradient across the essentially helical diameter and/or the backpressure along the channel length in order to produce stable Dean vortical structures when evaluated at a Reynolds number from about 100 to 500. In such embodiments, the essentially helical shaped flow channels are preferably dimensioned and arranged to increase heat-transfer and/or mass-transfer performance through formation of stable Dean vortical structures due to the winding number, pressure gradient, and/or backpressure, preferably wherein the stable Dean vortical structures are most operative under non-turbulent flow conditions, thereby creating secondary flow, lateral to a longitudinal channel base flow, and enhancing interactions with channel walls.

In other embodiments, the capture device substrate comprises a plurality of flow channels, preferably a plurality of identically-sized flow channels formed along a longitudinal axis of symmetry of the substrate body, wherein the flow channels have channel centerlines that are non-coincident with each other, and wherein each of the flow channel is configured into an essentially helical-essentially sinusoidal shape having a selected essentially helical diameter (radius), a channel length; and a pitch or winding number of essentially helical turns which is independent of the channel length, and the winding number is selected to optimize a pressure gradient across the essentially helical diameter and/or the backpressure along the channel length in order to produce stable Dean vortical structures when evaluated at a Reynolds number from about 100 to 500. In embodiments, the dimensions and arrangement of the essentially helical-essentially sinusoidal channels within the substrate is adapted to increase heat-transfer and/or mass-transfer performance through formation of stable vortical structures due to the selected winding number, pressure gradient and/or backpressure, such that the stable Dean vortical structures are operative under non-turbulent flow conditions, and which create secondary flow, lateral to a longitudinal channel base flow, thereby enhancing interactions with the channel walls.

In other embodiments, a capture device substrate comprises a plurality of flow channels, preferably identically-sized flow channels, formed along a longitudinal axis of symmetry of the substrate, wherein the flow channels have channel centerlines that are non-coincident with each other, and wherein each of the flow channel is configured into a essentially sinusoidal-essentially helical arrangement having a selected essentially helical diameter, a selected channel length; and a selected winding number of essentially helical turns independent of the channel length, and wherein the winding number is selected in order to optimize a pressure gradient across the essentially helical diameter and/or the backpressure along the given channel length in order to produce stable Dean vortical structures preferably the essentially sinusoidal-essentially helical channels are dimensioned and arranged to increase heat-transfer and/or mass-transfer performance through formation of stable vortical structures due to the winding number, pressure gradient, and/or backpressure, wherein the stable Dean vortical structures are most efficiently operative under non-turbulent flow conditions, thereby creating secondary flow, lateral to a longitudinal channel base flow, and enhancing interactions with channel walls.

In other embodiments, the capture device substrate comprises a body comprising a plurality of essentially sinusoidal shaped flow channels (flow channels having an essentially sinusoidal shaped flow path) formed therein along a longitudinal axis of symmetry of the substrate body. In embodiments, each of the essentially sinusoidal shaped flow channels, having an inlet opening separated from an outlet opening by a substrate length, and further comprising a essentially sinusoidal amplitude, and a essentially sinusoidal wavelength configured to increase heat-transfer and/or mass-transfer performance through formation of stable Dean vortical structures, which are most efficaciously operative under non-turbulent flow conditions, which create secondary flow within a fluid flowing through each of the essentially sinusoidal shaped channels, lateral to a longitudinal channel base flow through each of the essentially sinusoidal channels, and enhance interactions of the fluid flowing therethrough with channel walls.

In some embodiments, the channels of the capture device substrate are circular, square, rectangular, polygonal, wavy, essentially sinusoidal, and/or triangular.

In embodiments, the capture device substrate is formed from a ceramic material. In other embodiments, the capture device substrate comprises at least one metal and/or a polymeric (thermoplastic polymers, thermoset polymers), and may further include or be at least partially formed from a sorbent material.

### Formation of the Substrate

At least a portion of the capture device substrates according to embodiments disclosed herein may be manufactured out of ceramics, metals, thermoplastic polymers, thermoset polymers, or a combination thereof. In embodiments, the capture device substrate body or core may be produced via extrusion molding. According to one or more embodiments, a process for manufacturing ceramic linear and non-linear channels includes extrusion of the soft (uncured or green) ceramic materials whose composition is carefully controlled. The ceramic is extruded through a die outlet having a pattern which produces the flow channels, e.g., a thin mesh or lattice, which results in the formation of the flow channels. In embodiments, the die is moved relative to the extruder output to form the channels as described herein. After extrusion, the extrudate is trimmed to a length appropriate for a catalyst application and heat cured to produce the capture device substrate. In some embodiments, the heat-cured capture device substrate is contacted with a catalyst, typically via washcoat, according to methods known in the art. The capture device substrate may then be mounted and packaged in a housing or shell.

In other embodiments, at least a portion of the capture device substrate may be produced by additive manufacturing, e.g., 3-D printing. This includes ceramics, metals, thermoplastic polymers, thermoset polymers, or a combination thereof. For example, using a process referred to in the art as binder jetting, a polymeric or other type of sorbent may be directly printed to form at least a portion of the direct capture substrate. In other embodiments, at least a portion of the direct capture substrate comprises a support material such as mesoporous silica or mesoporous alumina, which is produced to a rigid support, e.g., sintered or cured, and then functionalized with a sorbent material, such as polyethyleneimine (PEI), via wet impregnation, incipient wetness, and/or the like. This allows for a reduction in the thermal mass of the direct capture substrate relative to a baseline contactor that is formed from an inert material, such as a ceramic, and then coated with a sorbent/support material e.g., washcoated.

In some embodiments, the direct capture substrate is produced using materials and conditions selected to control the pore size, pore structure, and pore size distribution of the substrate material as well as the loading of sorbent mass on and/or within the substrate support material, internal mass transfer resistance may be decreased, further increasing the rate of transfer of CO₂ to the sorbent in the substrate.

In embodiments, the formation of substrates having essentially helical channels may comprise the step of rotating the die at a given angular velocity along its longitudinal axis of symmetry to produce the capture device substrate having essentially helical channels disposed along a central axis parallel to the center axis of the substrate. The rotation of the die causes the extruded soft ceramic or thermoplastic material to form thin, narrow, long, and identically-sized tube-like channels wound along the die's longitudinal axis of symmetry in a manner similar to a helix. The speed of the rotation of the die is selected to produce the requisite number of essentially helical turns per given substrate length.

In an alternative embodiment, to form a capture device substrate having essentially sinusoidal channels, the die is oscillated along a vertical axis and/or a horizontal axis relative to the extruder output according to the amplitude and arrangement of the sine-wave or sinusoids to be formed in substrate. The specified frequency and mass-output of the extruder are controlled to form thin, narrow, long, essentially sinusoidal channels or cells that rise and fall along the die's longitudinal axis of symmetry according to a sinusoid function. In yet another embodiment, capture device substrates having essentially helical sinusoids and essentially sinusoidal helices may be formed by both oscillating along one or more axes, and/or rotation in one or more directions relative to the extruder output. The frequency and angular speed of die's essentially sinusoidal motion, and the speed of its rotation, will determine wavelength, amplitude, and essentially helical turns for any specified design.

In other embodiments, the extrudate flows through the die and into a form which supports the extrudate. This form is then moved relative to the extruder output i.e., via oscillation along one or more axes, rotations along one or more axes, or a combination thereof to form the channels according to embodiments disclosed herein, followed by curing of the ceramic to form the capture device substrates according to embodiments disclosed herein.

The reaction substrate may be formed out of any suitable ceramic known in the art. Likewise, in embodiments, the capture device substrate may be formed from materials which further include one or more catalytic materials such that the capture device substrates comprise one or more catalytic materials disposed within the wall of the flow channels. Suitable ceramic materials include those disclosed in U.S. Pat. Nos. 3,489,809, 5,714,228, 6,162,404, and 6,946,013, the content of which are fully incorporated by reference herein.

In other embodiments, the capture device substrates are formed essentially from metal, preferably metal sheeting, or foil. In an embodiment, the metallic substrate is manufactured into a conventional shape with straight and parallel tube-like channels, and then essentially helically twisted into a suitable essentially helical shape. In other embodiments, the manufacture of a metallic substrate core with essentially sinusoidal-essentially helical channels comprising forming the metal sheet into an essentially sinusoidal shape and stacking sheets into a block, followed by brazing or otherwise permanently affixing the sheets into place which may be followed by essentially helically twisting the formation to form essentially sinusoidal-essentially helical channels.

In other embodiments, the capture device substrates are formed essentially from thermoplastic polymers, thermoset polymers, or a combination thereof (plastic), preferably as a thin sheet. They may also be cast, injection molded, or 3D printed to produce the capture device substrate. In an embodiment, the plastic substrate is manufactured into a conventional shape with straight and parallel tube-like channels, and then essentially helically twisted into a suitable essentially helical shape. In other embodiments, the manufacture of a plastic substrate core with essentially sinusoidal-essentially helical channels comprising forming the metal sheet into an essentially sinusoidal shape and stacking sheets into a block, followed by welding or otherwise permanently affixing the sheets into place which may be followed by essentially helically twisting the formation to form essentially sinusoidal-essentially helical channels. In other embodiments, the direct capture substrate is formed by extrusion of the thermoplastic and/or thermoset polymer according to one or more methods by which ceramic substrates may be formed, as disclosed herein.

In one or more embodiments, a metallic and/or plastic capture device substrate may be manufactured from corrugated sheets folded first into a block, and then wound into a spiral, wherein a metal or plastic sheet is pressed or otherwise formed into a desirable corrugation, which is then formed into a channel shape. During this process, sheets of corrugated metal are stacked into blocks that are spirally wound and brazed, welded or permanently affixed into place. The blocks are then cut into individual substrate cores to form the channels. Once formed, the substrate may be washcoated with a slurry or solution comprising the catalyst and subsequently cured or fixed to bond or adhere the catalyst to the substrate.

In other embodiments, the capture device substrate may be formed by a process comprising three-dimensional (3-D) printing of the substrate, from metal, ceramic, plastic, or a combination thereof, and/or by forming a mold and casting the substrate.

3-D printing is suitable for manufacturing capture device substrates having essentially helical channels, essentially sinusoidal channels, essentially helical-sinusoid channels, and essentially sinusoidal essentially helical channels. Manufacture using 3-D printing comprises programing the printer with an appropriate computer-aided design (CAD) or digital model of a capture device substrate. Still other technologies, and methods of manufacturing capture device substrates according to one or more embodiments disclosed herein are suitable.

Accordingly, in embodiments, a method for manufacturing a ceramic capture device substrate comprises the steps of providing a die perforated with a lattice over an outlet of an extruder; extruding soft ceramic materials through the whilst the die is rotated along its axis of symmetry in a clockwise or counterclockwise manner in order to make a substrate having essentially helical channels of an essentially helical diameter, a channel length; and a winding number of essentially helical turns which is independent of the channel length. Preferably, the winding number is selected in order to optimize a pressure gradient across the selected essentially helical diameter and/or backpressure along the channel length in order to produce stable Dean vortical structures in a fluid flowing through the channel. In embodiments, the capture device substrate is adapted to increase heat-transfer and/or mass-transfer performance through formation of stable Dean vortical structures due to the winding number, pressure gradient, and/or backpressure, and further the channels are dimensioned and arranged to form stable Dean vortical structures which are exclusively operative under strictly non-turbulent flow conditions, to create secondary flow, lateral to a longitudinal channel base flow, and enhance interactions with channel walls. The method may further include trimming a plurality of the extruded substrates and heat curing and/or crosslinking, the substrates to form the capture device substrates.

In some embodiments the die is moved up and down along its axis of symmetry in order to superimpose an essentially sinusoidal channel into the essentially helical channel of the capture device substrate. In embodiments, the essentially sinusoidal waveforms formed in the channels are controlled by selecting a substrate length and selecting a frequency, amplitude, and wavelength of the up-and-down motion of the die during the extrusion process.

In embodiments, the process further includes coating the capture device substrate with a washcoat that contains a sorbent formulation; and optionally installing the capture device substrate within a protective outer housing having a fluid inlet and a fluid outlet on opposite ends of the direct capture substrate through which the fluid enters and exits the housing.

In embodiments, the extrusion may further include controlling the winding number of essentially helical turns formed in the essentially helical substrate per a given substrate length by adjusting a frequency with which the die is rotated clockwise or counterclockwise around a center axis of the die, optionally combined with the up-and-down motion of the die.

In other embodiments, a method for manufacturing a metallic and/or plastic capture device substrate comprises the steps of pressing a sheet of the material into a corrugated pattern having a plurality of identically-sized flow channels formed along a longitudinal axis of the pressed sheet, stacking a plurality of said pressed sheets all oriented along their longitudinal axes, permanently affixing each of the pressed sheets to each other into a block; and trimming the block into a length suitable for a capture device substrate.

In some embodiments, the step of pressing the sheet forms identically-sized essentially helical grooves in a flow direction along the longitudinal axis of the pressed sheet in lieu of the corrugated pattern, wherein the identically-sized essentially helical grooves have groove axes that are non-coincident with each other, and wherein each of the identically-sized essentially helical groove have a selected essentially helical diameter, a selected channel length, and a selected winding number of essentially helical turns, independent of the channel length. In embodiments, the winding number is selected in order to optimize a pressure gradient across the essentially helical diameter and backpressure along the channel length in order to produce stable Dean vortical structures, preferably which are sized and adapted to increase heat-transfer and/or mass-transfer performance through formation of stable Dean vortical structures due to the winding number, pressure gradient, and/or backpressure, in which the stable Dean vortical structures are exclusively operative under strictly non-turbulent flow conditions, thereby creating secondary flow, lateral to a longitudinal channel base flow, and enhance interactions with channel walls.

In some embodiments, the method may further comprise the step of essentially helically twisting the block along a longitudinal axis of the block to form essentially helical grooves along the axis, wherein the essentially helical grooves have groove axes that are non-coincident with each other, and wherein each the essentially helical groove has a selected essentially helical diameter, a channel length, and a winding number of essentially helical turns independent of the channel length, which are preferably selected to optimize a pressure gradient across the essentially helical diameter and backpressure along the channel length in order to produce stable Dean vortical structures.

In other embodiments, a method for manufacturing a ceramic and/or plastic capture device substrate comprises the steps of providing the die perforated with a lattice over an outlet of an extruder, extruding the soften materials through said die whilst said die is moved up and down relative to its axis of symmetry of the die to form essentially sinusoidal shaped channels. The method may further include trimming and heat curing and wash coating as above. In such embodiments, the step of extruding may further include controlling a number of essentially sinusoidal waveforms formed in the substrate per substrate length by adjusting a frequency, the essentially sinusoidal amplitude, and/or the essentially sinusoidal wavelength of the up-and-down motion with which the die is moved.

In other embodiments, at least a portion of the capture device substrates are produced using additive manufacturing techniques.

Capture device substrates according to one or more embodiments disclosed herein provide improved efficiency of the sorbent due to the formation of Dean vortices and/or similar secondary flows. The flow channels have improved packing due to improved fitting of cross-sectional shapes selected from a group including square, rectangular, polygonal, and triangular.

Capture device substrates according to one or more embodiments disclosed herein provide improved cost savings since the enhanced efficiency allows for a reduction of substrate volume (downsizing), a reduction in the amount of sorbent and/or the like, which is of considerable economic importance since many sorbent formulations are expensive, particularly when their formulations include precious metals (platinum, palladium, and rhodium). Downsizing allows non-negligible, multi-layered savings in costs of: (a) substrate, (b) sorbent washcoat, (c) sorbent precious metal(s), (d) sorbent coating process, (e) substrate packaging and support materials, and the like.

Capture device substrates according to one or more embodiments disclosed herein provide improved energy utilization since reduced size results in less energy expenditure due to a reduction in backpressure, a reduction in pumping power, weight reduction, and improved sorbent performance.

When compared to capture device substrates having linear channels, the capture device substrates according to one or more embodiments disclosed herein comprise higher catalytic efficiencies, heat transfer, and the like. Essentially helical channels further provide improved residence time of the fluid to be treated since they are longer than comparative linear channels disposed within the same honeycomb length; and/or provide improved mass transport due to the Dean vortices and other flow patterns when compared to linear channels; and/or improved heat transfer or thermal dissipation due to these same factors.

Likewise, the capture device substrates disclosed herein are suitable for use in heat-exchangers, filters, and the like, wherein the shape, arrangement and other properties of the channels and the substrate are selected according to the operational conditions.

### Alternative Essentially Helical Flow Channels

In embodiments, a substrate comprises an inlet end separated from an outlet end by a body length, the inlet end in fluid communication with the outlet end through a plurality of essentially helical channels coaxially disposed through the body about a central axis of the body, each of the channels comprising a cross-sectional shape determined orthogonal to the body central axis having a plurality of sides and a channel centerline defined by a geometric center of the cross-section of the channel at each point along the body length from the inlet end to the outlet end, the plurality of channels dimensioned and arranged such that each of the channel centerlines are essentially equal in length.

In some embodiments, essentially helical channels comprise a radius R equal to a distance determined orthogonal to the central axis from the channel centerline to the central axis of a channel, and a pitch P equal to a length of the channel centerline through one complete rotation of the channel about the central axis according to the equation P=2πK such that the body length H=PN=2πKN, wherein N is the number of rotations of the channel about the central axis from the inlet end to the outlet end;

wherein the length of the channel centerline L is according to the equation:

L=2πN\sqrt{R²+K²};

a ratio of the length of the channel centerline L to the body length H is defined by the equation:

$$\frac{L}{H} = \frac{2 π N \sqrt{{R}^{2} + {K}^{2}}}{2 π N K} = \frac{\sqrt{{R}^{2} + {K}^{2}}}{K} ;$$

and
wherein the ratio

$$\frac{L}{H}$$

of each of the plurality of essentially helical channels is essentially equal.

In some embodiments, each of the channels has a cross-sectional shape comprising 3 or more sides, or 4 or more sides, or 5 or more sides, or 6 or more sides. In embodiments, the channels are dimensioned such that a fluid flowing from the inlet end to the outlet end at a flow rate consistent with an intended use of the substrate forms a plurality of secondary flows having a Dean vortex-type flow pattern within one or more of the channels. In embodiments, each of the channels has a cross-sectional shape comprising an infinite number of sides.

In one or more embodiments, at least one sides of a first channel forms at least a portion of a side of at least one other channel.

In embodiments, a process to form a capture device substrate comprises the step of extrusion, 3d-printing, or a combination thereof. In one or more embodiments, the capture device substrate is formed from one or more ceramics, metals, plastics (thermoplastic polymers, thermoset polymers) or a combination thereof. In embodiments, the substrate comprises a plurality of metal sheets disposed about the central axis. In embodiments, the substrate is formed from a plurality of metal sheets disposed about the central axis comprising a plurality of corrugated sheets oriented with respect to a centerline of the corrugations at an angle from about 5° to 85° relative to a center axis of the substrate, separated from one another by a corresponding number of flat sheets wherein contact between the corrugated sheet and the flat sheet forms the cross-sectional shape of the channels, and wherein the corrugated sheets are disposed about the central axis.

In an alternative embodiment, the substrate is formed from a plurality of metal and/or plastic sheets disposed about the central axis at an angle with respect to the corrugations, comprising a plurality of corrugated sheets having a first cross-sectional shape separated from one another by a corresponding number of corrugated sheets having a second cross-sectional shape, wherein contact between the corrugated sheets forms the cross-sectional shape of the channels, and wherein the corrugated sheets are disposed about the central axis.

In embodiments, the cross-sectional area of each channel is uniform throughout the channel from the inlet end to the outlet end.

In one or more embodiments, a substrate comprises an inlet end separated from an outlet end by a body length, the inlet end in fluid communication with the outlet end through a plurality of essentially helical channels each disposed through the body about a corresponding channel center axis, each of the channels comprising a cross-sectional area bound by a plurality of sides and determined orthogonal to the center axis at each point between the inlet end and the outlet end along the body length, wherein the cross-sectional area of each channel varies periodically between a minimum value and a maximum value along the center axis.

In embodiments, each of the plurality of channels have at least one sides in common with another of the plurality of channels separating the two channels. In embodiments, each of the common sides separating two channels has essentially the same thickness throughout.

In some embodiments, each channel has a cross-section having 6 sides. In alterative embodiments, each channel has a cross-section having 4 sides. In alterative embodiments, each channel has a cross-section having 3 sides.

In embodiments, each of the plurality of channels has at least one side in common with at least one adjacent channel such that no empty space is present between the channels.

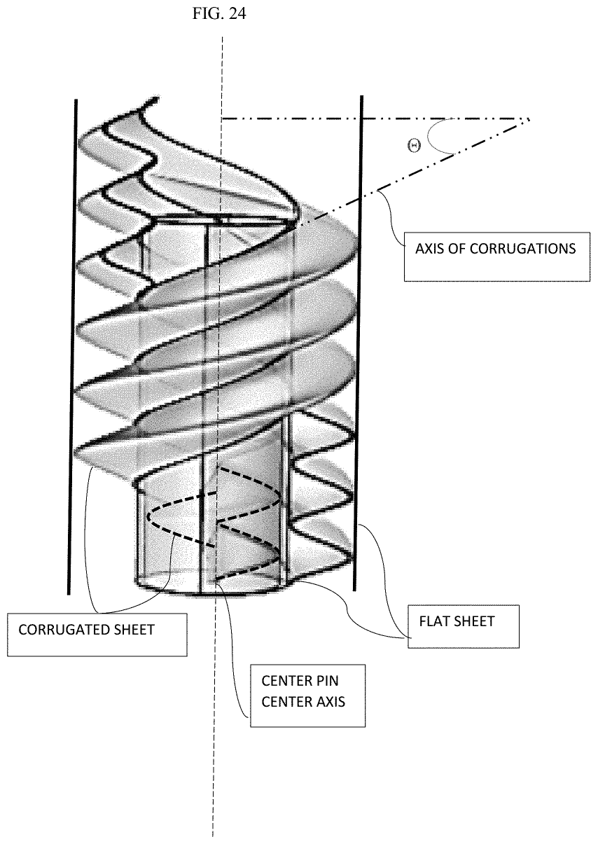

In embodiments, as shown in FIG. 24, the metallic and/or plastic capture device substrates comprise essentially helical channels and are manufactured from metallic foils or sheets and/or plastic sheets in an augmented manner. Manufacture of the essentially helical capture device substrates according to embodiments disclosed herein includes the steps of providing the corrugated sheets and then wrapping these sheets at an angle (Θ) to the central axis of the capture device substrate (the honeycomb axis of symmetry). Instead of being wound continuously outward from the center of the honeycomb according to common practice in the art. In embodiments, each layer is wound separately. In the minimal case as shown in FIG. 24, a corrugated sheet is wound around a central pin or tube forming channels between the tube and the corrugated sheet. Next, a flat sheet is wound around that formation forming channels between the other side of the corrugations and the flat sheet, followed by winding of additional alternating pairs of corrugated sheets followed by flat sheets until a desired honeycomb diameter is reached. The flat sheets and corrugated walls are then joined through brazing, spot-welding, or some other suitable technique. Accordingly, in embodiments, the capture device substrate is formed from a plurality of metal and/or plastic sheets disposed about the central axis comprising a plurality of corrugated sheets, oriented with respect to a centerline of the corrugation disposed into the sheet (e.g., along a fold line in the sheet) at an angle from about 5° to 85° relative to a center axis of the substrate. In embodiments, the corrugated substrates are separated from one another by a corresponding number of flat sheets wherein contact between the corrugated sheet and the flat sheet forms the cross-sectional shape of the channels.

In other embodiments, the substrate is formed from a plurality of metal and/or plastic sheets disposed about the central axis comprising a plurality of corrugated sheets having a first cross-sectional shape separated from one another by a corresponding number of corrugated sheets having a second cross-sectional shape, wherein contact between the corrugated sheets forms the cross-sectional shape of the channels. In one or more embodiments, the cross-sectional area of each channel is uniform throughout the channel from the inlet end to the outlet end.

In one or more embodiments of the invention, the length along the centerline of each channel in the substrate is kept the same. To achieve this, using a square essentially helical channel the shape of the channel is defined by two parameters: Radius (shown here as R), the normal distance from the axis of symmetry to the channel centerline; and pitch (shown here as 2πK), the distance the channel centerline traverses in the direction of the axis of symmetry in one complete rotation. In addition, a channel height H may be defined, which is the channel pitch multiplied by the number of rotations N, and thus H=2πKN. The channel height is also depicted as the distance between the open faces of the substrate. The distance traveled along the centerline of the channel, L, is given by the formula: L=2πN\sqrt{R²+K²} and the ratio of channel length to height (L/H) is thus

$$\frac{2 π N \sqrt{{R}^{2} + {K}^{2}}}{2 π NK} = \frac{\sqrt{{R}^{2} + {K}^{2}}}{K} .$$

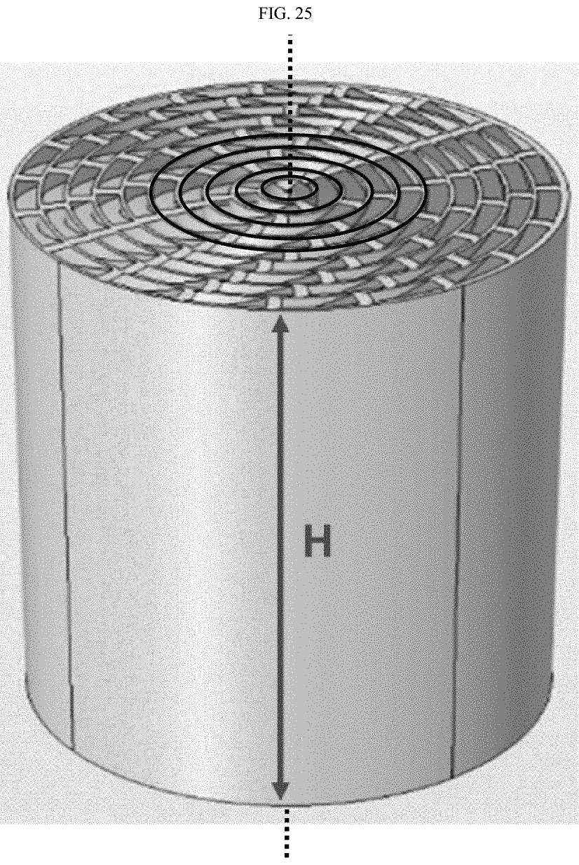

Applicant has discovered that by keeping this ratio the same for each channel in the substrate, each channel has the same length along the centerline for a given sorbent height. In other words, while the channels throughout the honeycomb vary in radius and pitch, if the ratio of pitch to radius of each channel is held the same, the channels have the same length along the centerline, given a honeycomb of uniform height. An example is shown in FIG. 25.

In such embodiments comprising essentially helical channels, each of the channels has a cross-sectional shape comprising 3 or more sides. In all embodiments, the channels are dimensioned such that a fluid flowing from the inlet end to the outlet end at a flow rate consistent with an intended use of the substrate forms a plurality of secondary flows having Dean vortices or Dean vortex-type flow patterns within the channels. In some embodiments, each of the channels has a cross-sectional shape comprising an infinite number of sides. In one or more embodiment, at least one sides of a first channel forms at least a portion of a side of a second channel. In one or more embodiments, the substrate is formed by extrusion, 3d-printing, or a combination thereof.

In one or more embodiments, the substrate is formed from one or more ceramics, metals, or a combination thereof. In other embodiments, the substrate is formed from a plurality of metal and/or plastic sheets radially disposed about the central axis.

In some embodiments, the substrate is formed from a plurality of metal and/or plastic sheets disposed about the central axis, the sheets comprising a plurality of corrugated sheets separated from one another by a corresponding number of flat sheets, wherein contact between the corrugated sheet and the flat sheet forms the cross-sectional shape of the channels.

### Variable Area/Radius Flow Channels

In an alternative embodiment, a substrate comprises an inlet end separated from an outlet end by a body length, the inlet end being in fluid communication with the outlet end through a plurality of essentially helical channels each disposed through the body about a corresponding center axis of the particular channel (a corresponding channel center axis), each of the channels comprising a cross-sectional shape bound by a plurality of sides and having a cross-sectional area determined orthogonal to the center axis of the channel at each point between the inlet end and the outlet end along the body length, wherein the cross-sectional area of each channel varies periodically between a minimum value and a maximum value along the center axis of the channel.

In one or more embodiments, the plurality of channels is arranged such that each channel has at least one side in common with another of the plurality of channels which separates the two channels from each other. In some of such embodiments, each of the common sides separating two channels is of essentially uniform thickness at each point along the center axis of the channel. In embodiments, the cross-sectional shape of the channels has greater than or equal to 3 sides. In some embodiments the cross-sectional shape of the channels has 6 sides. In other embodiments the cross-sectional shape of the channels has 4 sides. In some embodiments, each of the sides forming the cross-sectional shape are linear. In alterative embodiments, one or more of the sides forming the cross-sectional shape are non-linear, e.g., wavy, essentially sinusoidal, convex, concave, or any combination thereof. In some embodiments, each of the sides forming the cross-sectional shape are essentially linear and of equal length, e.g., the cross-sectional shape is a regular polygon. In alternative embodiments, one or more of the sides forming the cross-sectional shape have a different length than another of the sides, e.g., the cross-sectional shape is an irregular polygon.

In embodiments the plurality of channels is arranged to have at least one side in common with a neighboring channel such that no empty space is present between the channels.

In embodiments, each of the channels have at least one channel wall that separates a portion of two neighboring channels; each of these channel walls have essentially the same thickness, and the channels are arranged within the substrate such that an area occupied by the channels and the corresponding channel walls is greater than or equal to about 99% of the total area present in the substrate.

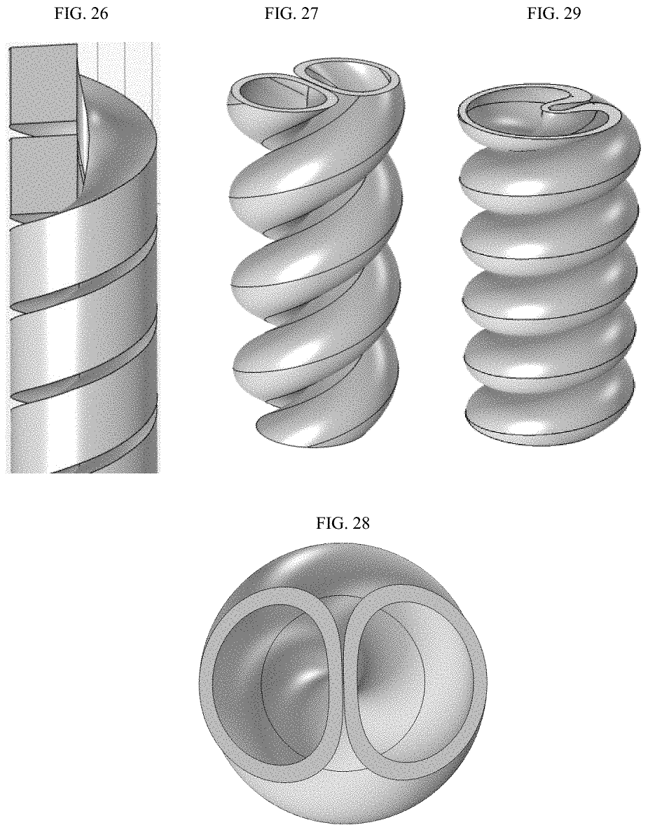

In embodiments, as shown in FIG. 26, one or more essentially helical channels may be nested within each other such that the essentially helical channels have a common center axis and one or more of the sides are common between two channels. FIG. 27 shows nested coaxial flow channels having an essentially round cross-sectional shape. FIG. 28 shows a cross-section of an alternative embodiment wherein the nested coaxial flow channels have a common wall between the two channels.

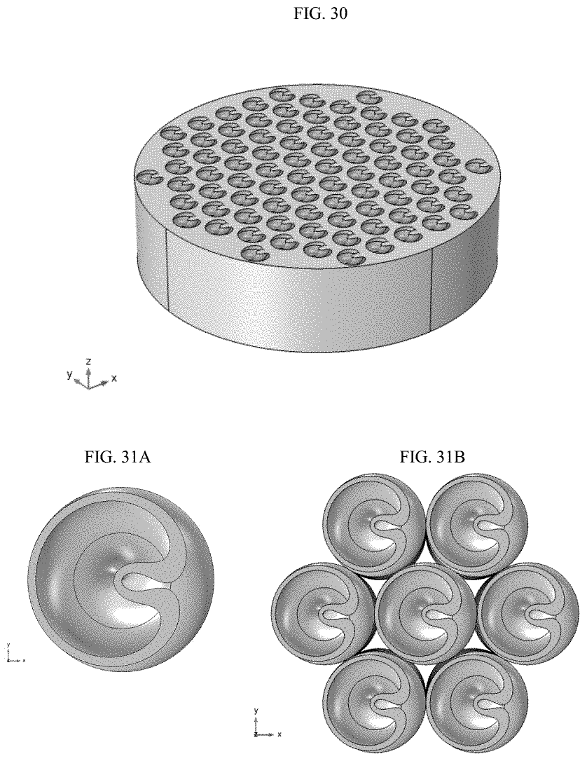

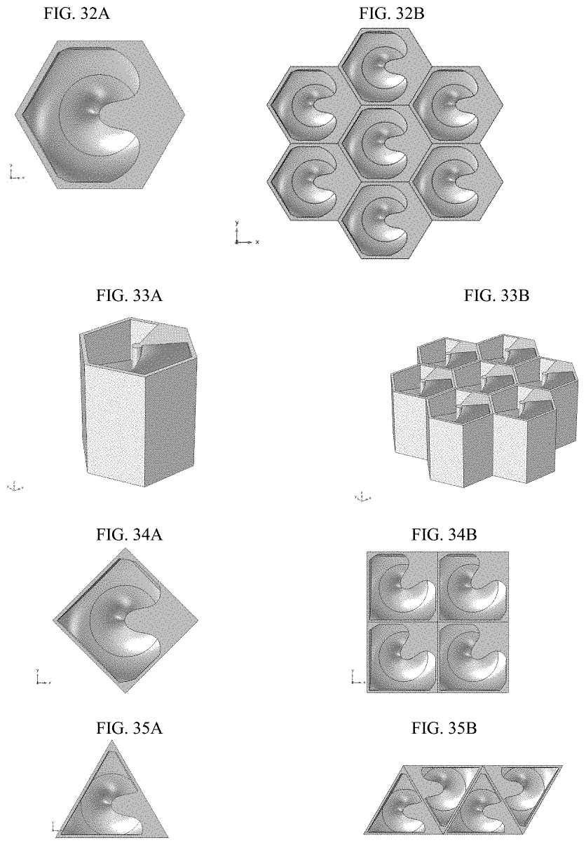

FIG. 29 shows an essentially helical channel according to an embodiment, FIG. 30 shows a plurality of the essentially helical channels disposed within a substrate. FIG. 31A shows an essentially helical flow channel having a circular cross-sectional shape. As shown in FIG. 31B, arrangement of circular essentially helical flow channels of equal cross-sectional area results in unused or wasted space between the flow channels. Indeed, the optimal packing of such circular flow channels results in less than 95% of the available space being utilized. As shown in FIGS. 32A and 32B, however, applicant has discovered that proper selection of the cross-sectional shape of the flow channel, in this case hexagonal, allows for essentially 100% packing efficiency of the flow channels within the capture device substrate. By utilizing a regular polygonal cross-sectional shape, each pair of flow channels has at least one common wall between the two, and as shown in FIGS. 33A and 33B, the walls are of uniform thickness and can be further minimized to reduce the thermal mass of the substrate while increasing the available surface area of the flow channel available for interaction with the fluid flowing therethrough. FIGS. 34A and 34B show the same type of packing available using flow channels with a square cross-sectional shape. FIGS. 35A and 35B show the same type of packing available using flow channels having a triangular cross-sectional shape.

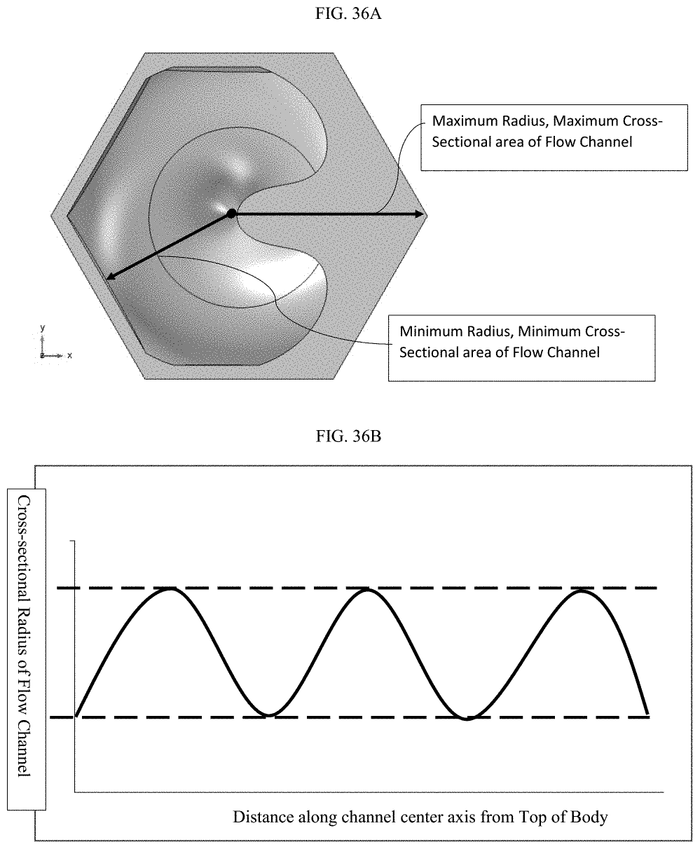

As shown in FIG. 36A, when the flow channel has a regular polygonal cross-sectional shape, in this case a hexagon, the radius of the channel determined between the center axis of the channel and the side wall, varies depending on the point longitudinally along the center axis at which the radius is determined. The minimum radius of the flow channel occurs at the center point of a linear flow channel wall and the maximum radius occurs at the intersection of two the of flow channel walls. FIG. 36B is a plot of the flow channel radius vs. the distance from the top of the body of the capture device substrate (the point along the center axis of the flow channel. As is shown, in such embodiments, the cross-sectional radius and thus the cross-sectional area of each channel varies periodically between a minimum value and a maximum value along the center axis.

Direct Capture Substrates having Permeable Flow Channels

In some embodiments, the capture device substrate comprises a first flow channel disposed proximate to a second flow channel, wherein at least a portion of at least one side of the first flow channel forms at least one common sidewall between at least a portion of at least one side of the second flow channel. In some of such embodiments, the capture device substrate includes at least a portion of the at least one common sidewall comprises a porosity, a conduit, a via, or a combination thereof, wherein the fluid inlet is in fluid communication with the fluid outlet through at least a portion of the at least one common sidewall.

In some embodiments, substrate includes inlet channels which are open on the inlet end of the substrate and in direct fluid communication with the fluid inlet of the capture device, and which are blocked on the outlet end of the substrate and thus not in direct fluid communication with the fluid outlet of the capture device. Adjacent to these inlet channels are disposed outlet channels which are closed on the inlet end of the substrate and thus not in direct fluid communication with the fluid inlet of the capture device, and which are open on the outlet end of the substrate and thus in direct fluid communication with the fluid outlet of the capture device. The inlet of the capture device is in fluid communication with the outlet of the capture device through the sidewalls of the inlet flow channels and the outlet flow channels.

This fluid communication between the inlet and the outlet of the capture device may include a porosity of the channel walls, via or holes disposed through the channel walls from an inlet channel to an outlet channel, valves or other gating mechanisms, or any combination thereof.

In an embodiment, the capture device substrate comprises a body having an inlet end separated longitudinally from an outlet end by a body length; a plurality of flow channels comprising a plurality of inlet flow channels and a plurality of outlet flow channels, each of the flow channels disposed into the body along a longitudinal axis and each bound by three or more sidewalls defining a cross-sectional shape and a cross-sectional area of the flow channel oriented orthogonal to the longitudinal axis; the inlet flow channels open on the inlet end and closed on the outlet end, and the outlet flow channels closed on the inlet end and open on the outlet end; the flow channels arranged within the body such that at least a portion of each inlet flow channel is in fluid communication with at least one outlet flow channel through at least a portion of at least one sidewall of the inlet flow channel having a porosity.

In some embodiments, the channel walls are further coated with and/or comprise and/or are formed at least partially from, one or more sorbents. The sorbents can include one or more catalytically active materials to impact the reaction rate of chemical reactions which consume species present in the fluid stream including particulates present therein, typically via oxidation from carbon to carbon dioxide which may be subsequently retained by the sorbent and water, in which the essentially helical shape and/or the essentially sinusoidal shape of the flow channels and the Dean vortices produced thereby further influence the reactions, or further influence the distribution, deposition, filtration or collection of the target species by the flow or by the porous wall.

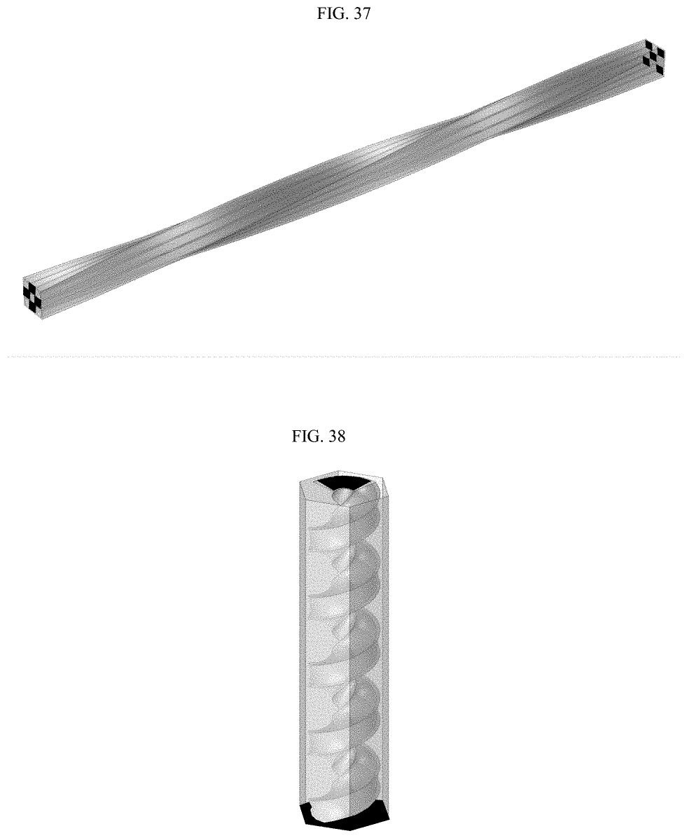

FIG. 37 shows an embodiment of the invention with channels comprising a unit cell or an entire substrate, wherein the channels alternate between being blocked at the inlet and outlet end, such that flow enters the channels that are plugged at the outlet end, flows through the walls of the substrate, and exits through the channels that are blocked at the inlet end with the channels formed along essentially helical paths around a common axis of symmetry and in which the centerline of the most central channel of the substrate is coincident with a common axis of symmetry.

FIG. 38 shows another embodiment, referred to a s candlestick design, in which the flow channels comprise a substrate wherein each inlet flow channel is blocked at the outlet end, is formed along an essentially helical path around its own axis of symmetry, such that the flow enters the individual inlet essentially helical channels and exits through the walls into the space around the channel which form the outlet flow channel which is blocked on the inlet end but open on the outlet end, where the flow is then directed to the substrate outlet by a circularly-shaped enclosure surrounding each channel individually.

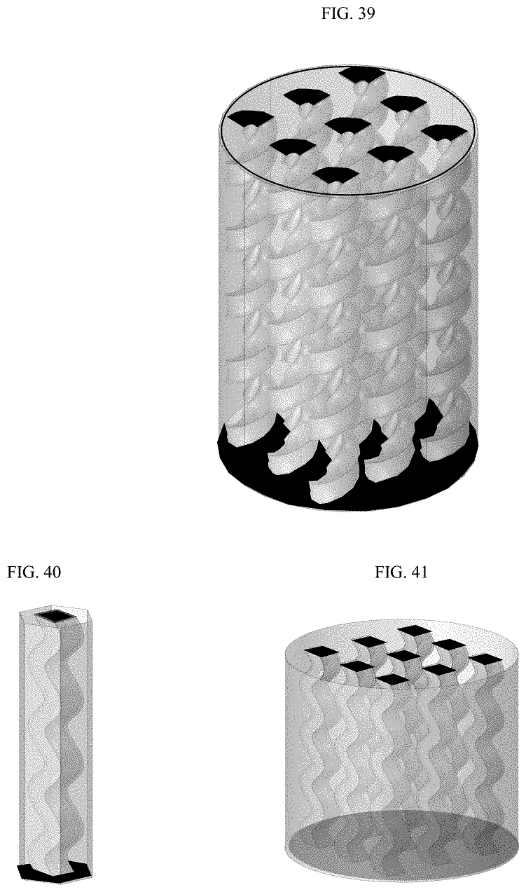

The hexagonal cross-sectional shape of the outlet channel shown in FIG. 38 allows for packing of the flow channels together, wherein there is essentially no wasted space between the channels. FIG. 39 shows another embodiment in which the plurality of flow channels comprise a substrate wherein each inlet flow channels, blocked at the outlet end, are formed within and along a longitudinal axis of the body, each of the inlet essentially helical flow channels have a flow path around its own axis of symmetry, such that the flow enters the individual essentially helical channels and exits through the walls into the space around the common outlet flow channel where it is then directed to the substrate outlet by an enclosure common to all outlet channels.

In embodiments, parameters of the essentially helical channels, namely radius of curvature, the pitch of the essentially helical path, the cross-sectional shape, the cross-sectional area of each flow channel and/or a combination thereof, is selected to promote improved sorption of the target compound or species present by the flow through the porous sidewalls.

In embodiments, for a particular cross-sectional area and flow-path length, the radius of curvature and pitch of the essentially helical path are selected to promote the preferred backpressure of the capture device substrate. Likewise, these same parameters are selected to promote improved desorption of the capture device substrate and/or sorbent loading amount and distribution to impact the rate and efficiency of the sorbent and/or chemical reactions.

FIG. 40 shows another embodiment in which the flow channels comprise a substrate wherein each inlet flow channel is blocked at the outlet end, and is formed along a essentially sinusoidal path, such that the flow enters the individual essentially sinusoidal inlet flow channels and exits through the porous sidewalls into the space around the hexagonal cross-section shaped channel forming the outlet flow channel where the treated fluid it is then directed to the substrate outlet by the hexagonally-shaped outlet flow channel surrounding each inlet flow channel individually.

FIG. 41 shows another embodiment in which the essentially sinusoidal inlet flow channels comprise a substrate wherein each inlet flow channel, blocked at the outlet end, is formed along a essentially sinusoidal path, such that the flow enters the individual essentially sinusoidal inlet flow channels and exits through the sidewalls into the space around the channel which forms a common outlet flow channel where the treated fluid is then directed to the outlet by an enclosure common to all channels.

In embodiments, the parameters of the essentially sinusoidal path of the flow channels, namely amplitude and period of the essentially sinusoidal path, along with, or for a particular cross-sectional shape and/or cross-sectional area, are selected to promote an enhanced sorption by the porous sidewalls, the backpressure of the capture device substrate, the preferred regeneration and/or desorption and release of the target materials of the capture device substrate, and/or the preferred sorption loading amount and distribution to impact the rate and efficacy of the sorbent.

Commercially speaking, such embodiments may be used for one or more DAC or other processes, such as for reactions (e.g. heterogeneous catalytic reactions), or for filtration, such as of particulate matter, or for other processes, or for a combination thereof. For example, in industrial processes in which DAC may be employed, filtration may be used, for instance, for filtration of soot and ash (also commonly called particulate or particulate matter) from diesel engines (commonly referred to as a Diesel Particulate Filter or DPF), or from gasoline engines such as a Gasoline Direct Injection (GDI) engine or a Port Injection (PI) engine (commonly referred to as a Gasoline Particulate Filter, GPF, Four-Way Catalyst, or FWC), or from other engines or devices (of which are known to produce particulate), or may be used for filtration or storage of fuel constituents laden in engine exhaust such as catalytically active fuel additives, which may also be present in the fluid to be treated.

In embodiments, the porosity of the common sidewall of the flow channels has an average pore size of greater than or equal to about 30 micrometers, or greater than or equal to about 100 micrometers, or greater than or equal to about 500 micrometers, or greater than or equal to about 1000 micrometers, or greater than or equal to about 2000 micrometers (2 mm) depending on the intended use of the direct air capture device. In some embodiments, the porosity results from vias and/or holes, e.g., laser drilled holes, through the common sidewall of the flow channels. In some embodiments, only a portion of the common sidewall between two flow channels is porous or otherwise capable of providing fluid communication from the fluid inlet to the fluid outlet of the direct capture device. In such embodiments, the porous substrate or portion of the flow channel may be formed by stamping, laser drilling, abrading, and/or other processes known in the art. Likewise, the porous portion of the flow channel may be formed from another material, e.g., a ceramic material, which is attached to or coated over fenestrations in the metal and/or plastic sidewall.

### Embodiments

Accordingly, the instant disclosure relates to the following embodiments:
E1. A capture device substrate comprising:
   a fluid inlet in fluid communication with a fluid outlet through at least one flow channel disposed along at least one flow path disposed within a body;
   the flow channel comprising a cross-sectional shape comprising a plurality of sides defining a cross-sectional area, determined orthogonal to the flow path;
   at least a portion of the flow path comprising an essentially sinusoidal shape, an essentially helical shape, or a combination thereof, configured to produce one or more stable Dean vortical structures (Dean-like vortical structures, essentially Dean vortical structures) in a fluid flowing through the flow channel when determined at a Reynolds number from about 100 to 500; and
   a sorbent effective to absorb, adsorb, sequester, and/or undergo a chemical reaction with one or more components present in the fluid flowing through at least a portion of the flow channel.
E2. The capture device substrate according to Embodiment E1, comprising a first flow channel disposed proximate to a second flow channel, wherein at least a portion of at least one side of the first flow channel forms at least one common sidewall between at least a portion of at least one side of the second flow channel.
E3. The capture device substrate according to Embodiment E1 or E2, wherein at least a portion of the at least one common sidewall comprises a porosity, a conduit, a via, or a combination thereof, wherein the fluid inlet is in fluid communication with the fluid outlet through at least a portion of the at least one common sidewall.
E4. The capture device substrate according to Embodiment E2 or E3, wherein the first flow channel is open on an inlet end of the body in direct fluid communication with the fluid inlet and closed on an outlet end of the body, and the second flow channel is closed on the inlet end of the body and open on an outlet end of the body in direct fluid communication with the fluid outlet.
E5. The capture device substrate according to any one of Embodiments E1 through E4, wherein at least a portion of the flow path comprises an essentially sinusoidal shape comprising an amplitude and a wavelength configured to produce the stable Dean vortical structures in the fluid flowing through at least a portion of the flow channel.
E6. The capture device substrate according to any one of Embodiments E1 through E5, wherein at least a portion of the flow path comprises an essentially helical shape oriented radially about a center axis of the flow channel and comprising a radius and a pitch configured to produce the stable Dean vortical structures in a fluid flowing through at least a portion of the flow channel.
E7. The capture device substrate according to any one of Embodiments E1 through E6, wherein at least a portion of the flow path comprises an essentially helical shape radially arranged about an essentially sinusoidal shape and comprising an amplitude, a wavelength, a radius and a pitch configured to produce the stable Dean vortical structures in the fluid flowing through at least a portion of the flow channel.
E8. The capture device substrate according to any one of Embodiments E1 through E7, wherein the flow path comprises an essentially sinusoidal shape arranged within an essentially helical shape oriented radially about a center axis of the flow channel, comprising an amplitude, a wavelength, a radius and a pitch configured to produce the stable Dean vortical structures in the fluid flowing through at least a portion of the flow channel.
E9. The capture device substrate according to any one of Embodiments E1 through E8, wherein at least a portion of the body comprises a plurality of flow channels, at least a portion of the plurality of flow channels comprising a flow path comprising a helical shape coaxially disposed about a single axis of the plurality of flow channels, each of the plurality of flow channels comprising a flow channel centerline defined by a geometric center of the cross-sectional shape of the flow channel at each point along a length of the portion of the body, the flow paths of each of the plurality of flow channels dimensioned and arranged within the portion of the body such that each of the flow channel centerlines are essentially equal in length.
E10. The capture device substrate according to any one of Embodiments E1 through E9, wherein at least a portion of the body comprises a plurality of flow channels, at least a portion of the plurality of flow channels comprising a flow path comprising an essentially helical shape coaxially disposed about a center axis of the corresponding flow channel,
   wherein the cross-sectional area of the flow channel varies periodically between a minimum value and a maximum value when determined along the center axis of the flow channel.
E11. The capture device substrate according to any one of Embodiments E1 through E10, wherein at least one flow channel has a cross-sectional shape comprising 3 or more sides.
E12. The capture device substrate according to any one of Embodiments E1 through E11, wherein at least a portion of the substrate is formed from one or more ceramics, metals, sorbents, thermoplastic polymers, thermoset polymers, or a combination thereof.
E13. The capture device substrate according to any one of Embodiments E1 through E12, formed from one or more metal sheets, polymeric sheets, or a combination thereof, disposed about at least one axis of the body.
E14. The capture device substrate according to Embodiment E13, wherein at least a portion of the substrate comprises a plurality of corrugated sheets separated from one another by a corresponding number of flat sheets wherein contact between the corrugated sheet and the flat sheet forms the cross-sectional shape of the flow channels;
   a plurality of corrugated sheets having a first cross-sectional shape separated from one another by a corresponding number of corrugated sheets having a second cross-sectional shape, wherein contact between the corrugated sheets forms the cross-sectional shape of the flow channels;
   or a combination thereof.
E15. The capture device substrate according to any one of Embodiments E1 through E14, wherein the body comprises an inlet end in fluid communication with the fluid inlet, and an outlet end in fluid communication with the fluid outlet, and wherein the cross-sectional area of each flow channel disposed within the body is essentially uniform from the inlet end to the outlet end of the body.
E16. The capture device substrate according to any one of Embodiments E1 through E15, wherein the sorbent is effective to absorb, adsorb, sequester, and/or undergo a chemical reaction with carbon dioxide.
E17. The capture device substrate according to any one of Embodiments E1 through E16, wherein the substrate is at least partially constructed from the sorbent and/or the substrate is functionalized with the sorbent.
E18. The capture device substrate according to any one of Embodiments E1 through E17, wherein the sorbent is present in a liquid, gel and/or slurry mobile phase flowing through one or more of the plurality of channels counter-current to the fluid flowing therethrough.
E19. The capture device substrate according to any one of Embodiments E1 through E18, wherein the sorbent is present in a liquid phase flowing through one or more of the plurality of channels counter-current to the fluid flowing therethrough, and wherein the sorbent is directed into the one or more flow channels through one or more channels laterally disposed into the body at an angle to the center axis of the body.
E20. A capture device comprising a capture device substrate according to any one of Embodiments E 1 through E19.
E21. The capture device according to Embodiment E20, comprising: a capture device substrate comprising a fluid inlet in fluid communication with a fluid outlet through at least one flow channel disposed along at least one flow path disposed within a body;
   the flow channel comprising a cross-sectional shape comprising a plurality of sides defining a cross-sectional area, determined orthogonal to the flow path;
   at least a portion of the flow path comprising an essentially sinusoidal shape, an essentially helical shape, or a combination thereof, configured to produce one or more stable Dean vortical structures in a fluid flowing through the flow channel when determined at a Reynolds number from about 100 to 500; and
   a sorbent effective to absorb, adsorb, sequester, and/or undergo a chemical reaction with one or more components present in the fluid flowing through at least a portion of the flow channel.
E22. The capture device according to any one of Embodiments E20 through E21, comprising:
   a capture device substrate comprising a body having an inlet end separated from an outlet end by a body length, the inlet end in fluid communication with the outlet end through a plurality of flow channels longitudinally disposed through the body;
   each of the flow channels having a cross-sectional shape comprising a plurality of sides defining a cross-sectional area, determined orthogonal to a longitudinal axis of the body;
   each of the flow channels having a sinusoidal shape oriented longitudinally along the body and comprising a sinusoidal amplitude, and a sinusoidal wavelength configured to produce stable Dean vortical structures in a fluid flowing therethrough; and
   a sorbent disposed within at least a portion of the flow channels effective to absorb and/or adsorb one or more components present in the fluid flowing therethrough.
E23. The capture device according to any one of Embodiments E20 through E22, comprising:
   a capture device substrate comprising a body having an inlet end separated from an outlet end by a body length, the inlet end in fluid communication with the outlet end through a plurality of flow channels longitudinally disposed through the body;
   each of the flow channels having a cross-sectional shape comprising a plurality of sides defining a cross-sectional area, determined orthogonal to a longitudinal axis of the body;
   each of the flow channels having a helical shape oriented radially about a longitudinal axis of the body and comprising a helical radius, and a helical pitch configured to produce stable Dean vortical structures in a fluid flowing therethrough; and
   a sorbent disposed within at least a portion of the flow channels effective to absorb and/or adsorb one or more components present in the fluid flowing therethrough.
E24. The capture device according to any one of Embodiments E20 through E23, comprising:
   a capture device substrate comprising a body having an inlet end separated from an outlet end by a body length, the inlet end in fluid communication with the outlet end through a plurality of flow channels longitudinally disposed through the body;
   each of the flow channels having a cross-sectional shape comprising a plurality of sides defining a cross-sectional area, determined orthogonal to a longitudinal axis of the body;
   each of the flow channels having a helical shape radially arranged about a sinusoidal shape oriented longitudinally along the body and comprising a sinusoidal amplitude, a sinusoidal wavelength, a helical radius and a helical pitch configured to produce stable Dean vortical structures in a fluid flowing therethrough; and
   a sorbent disposed within at least a portion of the flow channels effective to absorb and/or adsorb one or more components present in the fluid flowing therethrough.
E25. The capture device according to any one of Embodiments E20 through E24, comprising:
   a capture device substrate comprising a body having an inlet end separated from an outlet end by a body length, the inlet end in fluid communication with the outlet end through a plurality of flow channels longitudinally disposed through the body;
   each of the flow channels having a cross-sectional shape comprising a plurality of sides defining a cross-sectional area, determined orthogonal to a longitudinal axis of the body;
   each of the flow channels having a sinusoidal shape arranged within a helical shape oriented radially about a longitudinal axis of the body and comprising a sinusoidal amplitude, a sinusoidal wavelength, a helical radius and a helical pitch configured to produce stable Dean vortical structures in a fluid flowing therethrough; and
   a sorbent disposed within at least a portion of the flow channels effective to absorb and/or adsorb one or more components present in the fluid flowing therethrough.
E26. The capture device according to any one of Embodiments E20 through E25, comprising:
   a capture device substrate comprising a body having an inlet end separated from an outlet end by a body length, the inlet end in fluid communication with the outlet end through a plurality of flow channels longitudinally disposed through the body;
   each of the flow channels having a cross-sectional shape comprising a plurality of sides defining a cross-sectional area, determined orthogonal to a longitudinal axis of the body;
   the plurality of flow channels comprising a helical shape coaxially disposed about a central axis of the body, each of the channels comprising a channel centerline defined by a geometric center of the cross-sectional shape of the channel at each point along the body length from the inlet end to the outlet end, the plurality of channels dimensioned and arranged such that each of the channel centerlines are essentially equal in length;
   each helical channel comprising a cross-sectional area, a helical radius, and a helical pitch configured to produce stable Dean vortical structures in a fluid flowing therethrough; and
   a sorbent disposed within at least a portion of the flow channels effective to absorb and/or adsorb one or more components present in the fluid flowing therethrough.
E27. The capture device according to any one of Embodiments E20 through E26, comprising:
   a capture device substrate comprising a body having an inlet end separated from an outlet end by a body length, the inlet end in fluid communication with the outlet end through a plurality of flow channels longitudinally disposed through the body;
   each of the flow channels having a cross-sectional shape comprising a plurality of sides defining a cross-sectional area, determined orthogonal to a longitudinal axis of the body;
   each of the flow channels comprising a helical shape coaxially disposed about a corresponding channel axis wherein the cross-sectional area of each channel varies periodically between a minimum value and a maximum value along the channel axis; each helical channel comprising a helical radius, and a helical pitch configured to produce stable Dean vortical structures in a fluid flowing therethrough; and
   a sorbent disposed within at least a portion of the flow channels effective to absorb and/or adsorb one or more components present in the fluid flowing therethrough.
E28. The capture device according to any one of Embodiments E20 through E27, wherein each of the channels has a cross-sectional shape comprising 3 or more sides.
E29. The capture device according to any one of Embodiments E20 through E28, wherein at least one sides of a first channel forms at least a portion of a side of a second channel.
E30. The capture device according to any one of Embodiments E20 through E29, wherein the capture device substrate is formed from one or more ceramics, metals, or a combination thereof.
E31. The capture device according to any one of Embodiments E20 through E30, wherein the capture device substrate is formed from a plurality of metal and/or plastic sheets disposed about the central axis.
E32. The capture device according to any one of Embodiments E20 through E31, wherein the capture device substrate is formed from a plurality of metal and/or plastic sheets disposed about the central axis comprising a plurality of corrugated sheets separated from one another by a corresponding number of flat sheets wherein contact between the corrugated sheet and the flat sheet forms the cross-sectional shape of the channels.
E33. The capture device according to any one of Embodiments E20 through E32, wherein the capture device substrate is formed from a plurality of metal and/or plastic sheets disposed about the central axis comprising a plurality of corrugated sheets having a first cross-sectional shape separated from one another by a corresponding number of corrugated sheets having a second cross-sectional shape, wherein contact between the corrugated sheets forms the cross-sectional shape of the channels.
E34. The capture device according to any one of Embodiments E20 through E33, wherein the cross-sectional area of each channel is uniform throughout the channel from an inlet end to an outlet end of the capture device substrate.
E35. The capture device according to any one of Embodiments E20 through E34, wherein the plurality of channels are arranged within the capture device substrate to have at least one side in common with a neighboring channel such that no empty space is present between the channels.
E36. The capture device according to any one of Embodiments E20 through E35, wherein the sorbent is effective to absorb and/or adsorb carbon dioxide.
E37. The capture device according to any one of Embodiments E20 through E36, wherein the sorbent is present in a liquid phase flowing through one or more of the plurality of channels counter-current to the fluid flowing therethrough.
E38. The capture device according to any one of Embodiments E20 through E37, wherein the sorbent is present in a liquid phase flowing through one or more of the plurality of channels counter-current to the fluid flowing therethrough, and wherein the sorbent is directed into the one or more flow channels through one or more channels laterally disposed into the body at an angle to the center axis of the body.
E39. The capture device according to any one of Embodiments E20 through E38, comprising:
   a capture device substrate comprising a body having an inlet end separated longitudinally from an outlet end by a body length;
   a plurality of flow channels comprising a plurality of inlet flow channels and a plurality of outlet flow channels, each of the flow channels disposed into the body along a longitudinal axis and each bound by three or more sidewalls defining a cross-sectional shape and a cross-sectional area of the flow channel oriented orthogonal to the longitudinal axis;
   the inlet flow channels open on the inlet end and closed on the outlet end, and the outlet flow channels closed on the inlet end and open on the outlet end;
   the flow channels arranged within the body such that at least a portion of each inlet flow channel is in fluid communication with at least one outlet flow channel through at least a portion of at least one sidewall of the inlet flow channel having a porosity;
   each inlet flow channel having a sinusoidal shape oriented along the longitudinal axis and comprising a sinusoidal amplitude and a sinusoidal wavelength configured to produce stable Dean vortical structures in a fluid flowing therethrough, a helical shape oriented radially about the longitudinal axis and comprising a helical radius and a helical pitch configured to produce stable Dean vortical structures in a fluid flowing therethrough, or a combination thereof.
E40. The capture device according to any one of Embodiments E20 through E39, further comprising one or more catalysts disposed in or on one or more flow channel sidewalls.
E41. The capture device according to any one of Embodiments E20 through E40, wherein the porosity of the inlet flow channel has an average pore size of greater than or equal to about 30 micrometers and less than or equal to about 2000 micrometers.
E42. A method to remove a target compound from a fluid comprising:
   directing the fluid comprising the target compound through a capture device according to any one of Embodiments E20 through E41 at a flow rate, a temperature, and for a period of time sufficient to remove the target compound.
E43. The method according to Embodiment E42, further comprising a desorption step wherein the capture device substrate is placed under conditions sufficient to release the target compound from the sorbent.
E44. The method according to Embodiment E42 or E43, wherein the fluid is air and the target compound includes carbon dioxide.

### Examples

Various helical geometry configurations were tested against a straight channel baseline in the one-dimensional model. The baseline contactor properties were selected based on the modeling work known in the art, with channel properties slightly modified to match an experimental setup.

### Experimental Design

The experiment was designed in cooperation with the University of Washington Environmental Health Laboratory (UW EHL). The apparatus utilized an upstream source of compressed air or nitrogen fed through a mass flow meter followed by a flow heater and finally the direct capture device, referred to as the honeycomb. The effluent from the honeycomb was routed to an FTIR for species concentration measurement. Pressure drop was measured by a differential pressure transducer connected to taps upstream and downstream of the honeycomb. Data from the pressure transducer was fed to a voltage data logger and recorded. Temperatures were measured at various locations, including in the upstream and downstream flow path of the gas (fluid) directed through the honeycomb, on the honeycomb surface, and in the honeycomb channel, using K-type thermocouples fed into a temperature data logger. The flow temperature during desorption is controlled by feeding the upstream gas temperature into a PID controller which turns the flow heater off and on by means of a solid-state relay or SSR.

The experimental procedure is as follows:

### Purging

The honeycomb was installed in a testing mount on the test apparatus.

Establish a feed of 9 L/min heated, pure N₂ (110° C., 115° C. max). Hold for one hour.

After one hour, monitor outlet CO₂ concentration.

When outlet CO₂ concentration drops to 10 PPM or lower: Stop heating the incoming N₂.

Flow N₂ at ambient temperature. Continue until all thermocouples are in equilibrium with inlet thermocouple (˜25° C.).

### Measure Air Tank Concentrations

Check CO₂ and moisture concentrations of air tank feed prior to connecting/flowing into honeycomb.

Adsorption

Begin flowing 9 L/min air from tank through the purged honeycomb.

Measure, record outlet CO₂ concentration, temperatures, and differential pressure.

Continue gas flow until outlet CO₂ concentration reaches steady state or air tank concentration.

### Desorption

Flow 25° C. N₂ at 9 L/min then turn on flow heater to flow 100° C. N₂.

Measure CO₂ concentration, all thermocouples, and differential pressure.

Once CO₂ concentration reaches 10 PPM at outlet, turn off flow heater.

Flow ambient temperature N₂ until all thermocouples reach ambient temperature.

### Repeat Adsorption and Desorption Step

The main benefit predicted by the model-based comparison is that, under the simulated conditions, the improved mass transfer afforded by helical channels allows for the same CO₂ capture rate to be met with a 36.5% reduction in both contactor volume and sorbent mass. This comes at the cost of ˜20% increase in pressure drop and therefore pumping power. Given the high relative cost of sorbent capital expense in the overall cost breakdown of DAC as known in the art, this allows for ˜30% reduction in the cost of DAC relative to a straight baseline. The potential cost savings depends on the choice of baseline configuration. The relative benefit seen from helical channels increases with increasing flow rate, increasing hydraulic diameter, and decreasing baseline channel length.

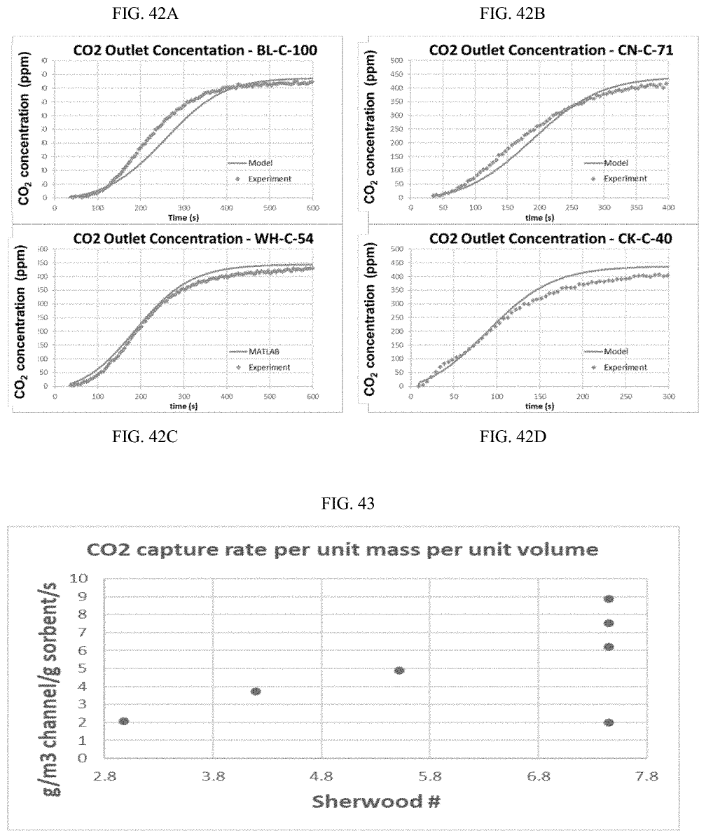

As the data in FIGS. 42 A-D and 43 show, helical channel monoliths would provide the ability to meet the same rate of mass transfer while reducing pressure drop due to their superior ratio of Sherwood number to friction factor. A small increase in pressure drop was also observed in one-dimensional models. This is likely due to increases in external mass transfer (from the bulk to the sorbent surface) being mitigated by internal mass transfer resistance and reduced concentration gradients as the sorbent fills with CO₂.

The substrates were evaluated in actual testing using a setup common in the art. Initial experimental results show good agreement with the model during adsorption (see FIGS. 42A-D). Early analysis of adsorption rates also demonstrated improvement (see FIG. 43).

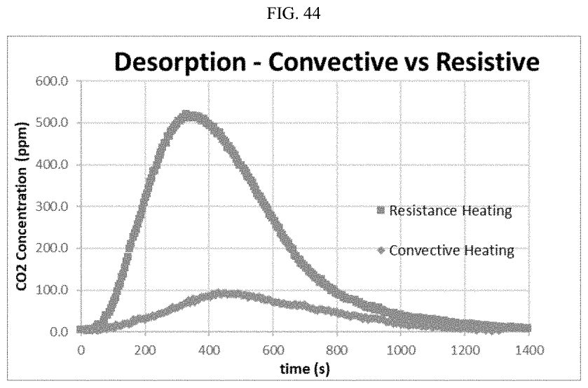

The testing further demonstrated the practicability of resistance heating of metallic honeycombs for desorption (see FIG. 44). As these data show, the use of resistance heating provides marked benefits over heating by convection.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs an essentially helical surface, in the environment of fastening wooden parts, a nail and a screw may be equivalent structures. It is the express intention of the applicant not to invoke 35 U.S.C. § 112, paragraph 6 for any limitations of any of the claims herein, except for those in which the claim expressly uses the words ‘means for’ together with an associated function.

We claim:

1. A capture device substrate comprising:
a fluid inlet in fluid communication with a fluid outlet through at least one flow channel disposed along at least one flow path disposed within a body;
the flow channel comprising a cross-sectional shape comprising a plurality of sides defining a cross-sectional area, determined orthogonal to the flow path;
at least a portion of the flow path comprising an essentially sinusoidal shape, an essentially helical shape, or a combination thereof, configured to produce one or more stable Dean vortical structures in a fluid flowing through the flow channel when determined at a Reynolds number from about 100 to 500; and
a sorbent effective to absorb, adsorb, sequester, and/or undergo a chemical reaction with one or more components present in the fluid flowing through at least a portion of the flow channel.

2. The capture device substrate of claim 1, comprising a first flow channel disposed proximate to a second flow channel, wherein at least a portion of at least one side of the first flow channel forms at least one common sidewall between at least a portion of at least one side of the second flow channel.

3. The capture device substrate of claim 2, wherein at least a portion of the at least one common sidewall comprises a porosity, a conduit, a via, or a combination thereof, wherein the fluid inlet is in fluid communication with the fluid outlet through at least a portion of the at least one common sidewall.

4. The capture device substrate of claim 3, wherein the first flow channel is open on an inlet end of the body in direct fluid communication with the fluid inlet and closed on an outlet end of the body, and the second flow channel is closed on the inlet end of the body and open on an outlet end of the body in direct fluid communication with the fluid outlet.

5. The capture device substrate of claim 1, wherein at least a portion of the flow path comprises an essentially sinusoidal shape comprising an amplitude and a wavelength configured to produce the stable Dean vortical structures in the fluid flowing through at least a portion of the flow channel.

6. The capture device substrate of claim 1, wherein at least a portion of the flow path comprises an essentially helical shape oriented radially about a center axis of the flow channel and comprising a radius and a pitch configured to produce the stable Dean vortical structures in a fluid flowing through at least a portion of the flow channel.

7. The capture device substrate of claim 1, wherein at least a portion of the flow path comprises an essentially helical shape radially arranged about an essentially sinusoidal shape and comprising an amplitude, a wavelength, a radius and a pitch configured to produce the stable Dean vortical structures in the fluid flowing through at least a portion of the flow channel.

8. The capture device substrate of claim 1, wherein the flow path comprises an essentially sinusoidal shape arranged within an essentially helical shape oriented radially about a center axis of the flow channel, comprising an amplitude, a wavelength, a radius and a pitch configured to produce the stable Dean vortical structures in the fluid flowing through at least a portion of the flow channel.

9. The capture device substrate of claim 1, wherein at least a portion of the body comprises a plurality of flow channels, at least a portion of the plurality of flow channels comprising a flow path comprising a helical shape coaxially disposed about a single axis of the plurality of flow channels, each of the plurality of flow channels comprising a flow channel centerline defined by a geometric center of the cross-sectional shape of the flow channel at each point along a length of the portion of the body, the flow paths of each of the plurality of flow channels dimensioned and arranged within the portion of the body such that each of the flow channel centerlines are essentially equal in length;
wherein at least a portion of the body comprises a plurality of flow channels, at least a portion of the plurality of flow channels comprising a flow path comprising an essentially helical shape coaxially disposed about a center axis of the corresponding flow channel, wherein the cross-sectional area of the flow channel varies periodically between a minimum value and a maximum value when determined along the center axis of the flow channel;
or a combination thereof.

10. The capture device substrate of claim 1, wherein at least one flow channel has a cross-sectional shape comprising 3 or more sides.

11. The capture device substrate of claim 1, wherein at least a portion of the substrate is formed from one or more ceramics, metals, sorbents, thermoplastic polymers, thermoset polymers, or a combination thereof.

12. The capture device substrate of claim 1, formed from one or more metal sheets, polymeric sheets, or a combination thereof, disposed about at least one axis of the body.

13. The capture device substrate of claim 12, wherein at least a portion of the substrate comprises a plurality of corrugated sheets separated from one another by a corresponding number of flat sheets wherein contact between the corrugated sheet and the flat sheet forms the cross-sectional shape of the flow channels;
a plurality of corrugated sheets having a first cross-sectional shape separated from one another by a corresponding number of corrugated sheets having a second cross-sectional shape, wherein contact between the corrugated sheets forms the cross-sectional shape of the flow channels;
or a combination thereof.

14. The capture device substrate of claim 1, wherein the body comprises an inlet end in fluid communication with the fluid inlet, and an outlet end in fluid communication with the fluid outlet, and wherein the cross-sectional area of each flow channel disposed within the body is essentially uniform from the inlet end to the outlet end of the body.

15. The capture device substrate of claim 1, wherein the sorbent is effective to absorb, adsorb, sequester, and/or undergo a chemical reaction with carbon dioxide.

16. The capture device substrate of claim 1, wherein the substrate is at least partially constructed from the sorbent and/or the substrate is functionalized with the sorbent.

17. The capture device substrate of claim 1, wherein the sorbent is present in a liquid, gel and/or slurry mobile phase flowing through one or more of the plurality of channels counter-current to the fluid flowing therethrough.

18. A capture device comprising a capture device substrate comprising:
a fluid inlet in fluid communication with a fluid outlet through at least one flow channel disposed along at least one flow path disposed within a body;
the flow channel comprising a cross-sectional shape comprising a plurality of sides defining a cross-sectional area, determined orthogonal to the flow path;
at least a portion of the flow path comprising an essentially sinusoidal shape, an essentially helical shape, or a combination thereof, configured to produce one or more stable Dean vortical structures in a fluid flowing through the flow channel when determined at a Reynolds number from about 100 to 500; and
a sorbent effective to absorb, adsorb, sequester, and/or undergo a chemical reaction with one or more components present in the fluid flowing through at least a portion of the flow channel.

19. A method to remove a target compound from a fluid comprising:
directing the fluid comprising the target compound through a capture device comprising a capture device substrate of claim 1 at a flow rate, a temperature, and for a period of time sufficient to remove the target compound.

20. The method of claim 19 further comprising a desorption step wherein the capture device substrate is placed under conditions sufficient to release the target compound from the sorbent.
